SEQUENCE LISTING (78 sequences)

SEQ ID NO: 1 - protein, 55 aa, Human Immunodeficiency virus 1
ARQLLSGIVQQQNNLLRAIEAQQHLLQLTVWGIKQLQARILAVERYLKDQQLLGI

SEQ ID NO: 2 - protein, 38 aa, Human Immunodeficiency virus 1
NNLLRAIEAQQHLLQLTVWGIKQLQARILAVERYLKDQ

SEQ ID NO: 3 - protein, 36 aa, Human Immunodeficiency virus 1
SGIVQQQNNLLRAIEAQQHLLQLTVWGIKQLQARIL

SEQ ID NO: 4 - protein, 56 aa, Human Immunodeficiency virus 1
WNNMTWMEWDREINNYTSLIHSLIEESQNQQEKNEQELLELDKWASLWNWFNITNW

SEQ ID NO: 5 - protein, 36 aa, Human Immunodeficiency virus 1
YTSLIHSLIEESQNQQEKNEQELLELDKWASLWNWF

SEQ ID NO: 6 - protein, 34 aa, Human Immunodeficiency virus 1
WMEWDREINNYTSLIHSLIEESQNQQEKNEQELL

SEQ ID NO: 7 - protein, 5 aa, Artificial Sequence
  Artificial linker peptide
GGGGS

SEQ ID NO: 8 - protein, 345 aa, Human Immunodeficiency virus 1
AVGIGALFLGFLGAAGSTMGARSMTLTVQARQLLSGIVQQQNNLLRAIEAQQHLLQLTVWGIKQLQARILAVERYLKDQQLLGIWGCSGKLICTTAVPWNASWSNKSLEQIWNNMTWMEWDREINNYTSLIHSLIEESQNQQEKNEQELLELDKWASLWNWFNITNWLWYIKIFIMIVGGLVGLRIVFAVLSIVNRVRQGYSPLSFQTHLPTPRGPDRPEGIEEEGGERDRDRSIRLVNGSLALIWDDLRSLCLFSYHRLRDLLLIVTRIVELLGRRGWEALKYWWNLLQYWSQELKNSAVSLLNATAIAVAEGTDRVIEVVQGACRAIRHIPRRIRQGLERILL

SEQ ID NO: 9 - protein, 45 aa, Human Immunodeficiency virus 1
SGIVQQQNNLLRAIEAQQHLLQLTVWGIKQLQARILAVERYLKDQ

SEQ ID NO: 10 - protein, 45 aa, Human Immunodeficiency virus 1
SGIVQQQNNLLRAIEAQQHLLQLTVWGIKQLQARVLALERYLRDQ

SEQ ID NO: 11 - protein, 36 aa, Human Immunodeficiency virus 1
SGIVQQQNNLLRAIEAQQHLLQLTVWGIKQLQARVL

SEQ ID NO: 12 - protein, 38 aa, Human Immunodeficiency virus 1
NNLLRAIEAQQHLLQLTVWGIKQLQARVLALERYLRDQ

SEQ ID NO: 13 - protein, 45 aa, Human Immunodeficiency virus 1
SGIVQQQNNLLRAIEAQQRMLQLTVWGIKQLQARVLAVERYLGDQ

SEQ ID NO: 14 - protein, 36 aa, Human Immunodeficiency virus 1
SGIVQQQNNLLRAIEAQQRMLQLTVWGIKQLQARVL

SEQ ID NO: 15 - protein, 38 aa, Human Immunodeficiency virus 1
NNLLRAIEAQQRMLQLTVWGIKQLQARVLAVERYLGDQ

SEQ ID NO: 16 - protein, 45 aa, Human Immunodeficiency virus 1
SGIVQQQNNLLRAIEAQQHMLQLTVWGIKQLQARVLALERYLRDQ

SEQ ID NO: 17 - protein, 36 aa, Human Immunodeficiency virus 1
SGIVQQQNNLLRAIEAQQHMLQLTVWGIKQLQARVL

SEQ ID NO: 18 - protein, 38 aa, Human Immunodeficiency virus 1
NNLLRAIEAQQHMLQLTVWGIKQLQARVLALERYLRDQ

SEQ ID NO: 19 - protein, 45 aa, Human Immunodeficiency virus 1
SGIVQQQSNLLRAIEAQQHMLQLTVWGIKQLQARVLAIERYLRDQ

SEQ ID NO: 20 - protein, 36 aa, Human Immunodeficiency virus 1
SGIVQQQSNLLRAIEAQQHMLQLTVWGIKQLQARVL

SEQ ID NO: 21 - protein, 38 aa, Human Immunodeficiency virus 1
SNLLRAIEAQQHMLQLTVWGIKQLQARVLAIERYLRDQ

SEQ ID NO: 22 - protein, 45 aa, Human Immunodeficiency virus 1
SGIVQQQNNLLRAIEAQQHLLQLTVWGIKQLQARVLAVESYLKDQ

SEQ ID NO: 23 - protein, 38 aa, Human Immunodeficiency virus 1
NNLLRAIEAQQHLLQLTVWGIKQLQARVLAVESYLKDQ

SEQ ID NO: 24 - protein, 45 aa, Human Immunodeficiency virus 1
SGIVQQQSNLLRAIEAQQHLLQLTVWGIKQLQARVLAVERYLQDQ

SEQ ID NO: 25 - protein, 36 aa, Human Immunodeficiency virus 1
SGIVQQQSNLLRAIEAQQHLLQLTVWGIKQLQARVL

SEQ ID NO: 26 - protein, 38 aa, Human Immunodeficiency virus 1
SNLLRAIEAQQHLLQLTVWGIKQLQARVLAVERYLQDQ

SEQ ID NO: 27 - protein, 45 aa, Human Immunodeficiency virus 1
SGIVQQQSNLLRAIEAQQHLLQLTVWGIKQLQARVLALERYLRDQ

SEQ ID NO: 28 - protein, 38 aa, Human Immunodeficiency virus 1
SNLLRAIEAQQHLLQLTVWGIKQLQARVLALERYLRDQ

SEQ ID NO: 29 - protein, 45 aa, Human Immunodeficiency virus 1
SGIVQQQSNLLRAIQAQQHMLQLTVWGVKQLQARVLAVERYLKDQ

SEQ ID NO: 30 - protein, 36 aa, Human Immunodeficiency virus 1
SGIVQQQSNLLRAIQAQQHMLQLTVWGVKQLQARVL

SEQ ID NO: 31 - protein, 38 aa, Human Immunodeficiency virus 1
SNLLRAIQAQQHMLQLTVWGVKQLQARVLAVERYLKDQ

SEQ ID NO: 32 - protein, 45 aa, Human Immunodeficiency virus 1
SGIVQQQSNLLKAIEAQQHLLKLTVWGIKQLQARVLAVERYLKDQ

SEQ ID NO: 33 - protein, 36 aa, Human Immunodeficiency virus 1
SGIVQQQSNLLKAIEAQQHLLKLTVWGIKQLQARVL

SEQ ID NO: 34 - protein, 38 aa, Human Immunodeficiency virus 1
SNLLKAIEAQQHLLKLTVWGIKQLQARVLAVERYLKDQ

SEQ ID NO: 35 - protein, 45 aa, Human Immunodeficiency virus 1
SGIVQQQNILLRAIEAQQHLLQLSIWGIKQLQAKVLAIERYLRDQ

SEQ ID NO: 36 - protein, 36 aa, Human Immunodeficiency virus 1
SGIVQQQNILLRAIEAQQHLLQLSIWGIKQLQAKVL

SEQ ID NO: 37 - protein, 38 aa, Human Immunodeficiency virus 1
NILLRAIEAQQHLLQLSIWGIKQLQAKVLAIERYLRDQ

SEQ ID NO: 38 - protein, 45 aa, Human Immunodeficiency virus 1
  misc_feature (25)..(25): Xaa can be any naturally occurring amino acid
KGIVQQQDNLLRAIQAQQQLLRLSXWGIRQLRARLLALETLLQNQ

SEQ ID NO: 39 - protein, 35 aa, Human Immunodeficiency virus 1
  misc_feature (25)..(25): Xaa can be any naturally occurring amino acid
KGIVQQQDNLLRAIQAQQQLLRLSXWGIRQLRARL

SEQ ID NO: 40 - protein, 38 aa, Human Immunodeficiency virus 1
  misc_feature (18)..(18): Xaa can be any naturally occurring amino acid
DNLLRAIQAQQQLLRLSXWGIRQLRARLLALETLLQNQ

SEQ ID NO: 41 - protein, 46 aa, Human Immunodeficiency virus 1
WMEWDREINNYTSLIHSLIEESQNQQEKNEQELLELDKWASLWNWF

SEQ ID NO: 42 - protein, 46 aa, Human Immunodeficiency virus 1
WMEWEREIENYTGLIYTLIEESQNQQEKNEQDLLALDKWASLWNWF

SEQ ID NO: 43 - protein, 34 aa, Human Immunodeficiency virus 1
WMEWEREIENYTGLIYTLIEESQNQQEKNEQDLL

SEQ ID NO: 44 - protein, 36 aa, Human Immunodeficiency virus 1
YTGLIYTLIEESQNQQEKNEQDLLALDKWASLWNWF

SEQ ID NO: 45 - protein, 46 aa, Human Immunodeficiency virus 1
WMEWEREIDNYTSEIYTLIEESQNQQEKNEQELLELDKWASLWNWF

SEQ ID NO: 46 - protein, 34 aa, Human Immunodeficiency virus 1
WMEWEREIDNYTSEIYTLIEESQNQQEKNEQELL

SEQ ID NO: 47 - protein, 36 aa, Human Immunodeficiency virus 1
YTSEIYTLIEESQNQQEKNEQELLELDKWASLWNWF

SEQ ID NO: 48 - protein, 46 aa, Human Immunodeficiency virus 1
WMEWEREIDNYTDYIYDLLEKSQTQQEKNEKELLELDKWASLWNWF

SEQ ID NO: 49 - protein, 34 aa, Human Immunodeficiency virus 1
WMEWEREIDNYTDYIYDLLEKSQTQQEKNEKELL

SEQ ID NO: 50 - protein, 36 aa, Human Immunodeficiency virus 1
YTDYIYDLLEKSQTQQEKNEKELLELDKWASLWNWF

SEQ ID NO: 51 - protein, 46 aa, Human Immunodeficiency virus 1
WIQWDREISNYTGIIYRLLEESQNQQENNEKDLLALDKWQNLWSWF

SEQ ID NO: 52 - protein, 34 aa, Human Immunodeficiency virus 1
WIQWDREISNYTGIIYRLLEESQNQQENNEKDLL

SEQ ID NO: 53 - protein, 36 aa, Human Immunodeficiency virus 1
YTGIIYRLLEESQNQQENNEKDLLALDKWQNLWSWF

SEQ ID NO: 54 - protein, 46 aa, Human Immunodeficiency virus 1
WMEWEREISNYTGLIYDLIEESQIQQEKNEKDLLELDKWASLWNWF

SEQ ID NO: 55 - protein, 34 aa, Human Immunodeficiency virus 1
WMEWEREISNYTGLIYDLIEESQIQQEKNEKDLL

SEQ ID NO: 56 - protein, 36 aa, Human Immunodeficiency virus 1
YTGLIYDLIEESQIQQEKNEKDLLELDKWASLWNWF

SEQ ID NO: 57 - protein, 46 aa, Human Immunodeficiency virus 1
WMEWQKEISNYSNEVYRLIEKSQNQQEKNEQGLLALDKWASLWNWF

SEQ ID NO: 58 - protein, 34 aa, Human Immunodeficiency virus 1
WMEWQKEISNYSNEVYRLIEKSQNQQEKNEQGLL

SEQ ID NO: 59 - protein, 36 aa, Human Immunodeficiency virus 1
YSNEVYRLIEKSQNQQEKNEQGLLALDKWASLWNWF

SEQ ID NO: 60 - protein, 46 aa, Human Immunodeficiency virus 1
WIQWDREISNYTQQIYSLIEESQNQQEKNEQDLLALDNWASLWTWF

SEQ ID NO: 61 - protein, 34 aa, Human Immunodeficiency virus 1
WIQWDREISNYTQQIYSLIEESQNQQEKNEQDLL

SEQ ID NO: 62 - protein, 36 aa, Human Immunodeficiency virus 1
YTQQIYSLIEESQNQQEKNEQDLLALDNWASLWTWF

SEQ ID NO: 63 - protein, 46 aa, Human Immunodeficiency virus 1
WMEWDRQIDNYTEVIYRLLELSQTQQEQNEQDLLALDKWDSLWNWF

SEQ ID NO: 64 - protein, 34 aa, Human Immunodeficiency virus 1
WMEWDRQIDNYTEVIYRLLELSQTQQEQNEQDLL

SEQ ID NO: 65 - protein, 36 aa, Human Immunodeficiency virus 1
YTEVIYRLLELSQTQQEQNEQDLLALDKWDSLWNWF

SEQ ID NO: 66 - protein, 47 aa, Human Immunodeficiency virus 1
WIQWEREINNYTGIIYSLIEEAQNQQENNEKDLLALDKWTNLWNWFN

SEQ ID NO: 67 - protein, 34 aa, Human Immunodeficiency virus 1
WIQWEREINNYTGIIYSLIEEAQNQQENNEKDLL

SEQ ID NO: 68 - protein, 37 aa, Human Immunodeficiency virus 1
YTGIIYSLIEEAQNQQENNEKDLLALDKWTNLWNWFN

SEQ ID NO: 69 - protein, 46 aa, Human Immunodeficiency virus 1
WQQWDEKVRNYSGVIFGLIEQAQEQQNTNEKSLLELDQWDSLWSWF

SEQ ID NO: 70 - protein, 34 aa, Human Immunodeficiency virus 1
WQQWDEKVRNYSGVIFGLIEQAQEQQNTNEKSLL

SEQ ID NO: 71 - protein, 36 aa, Human Immunodeficiency virus 1
YSGVIFGLIEQAQEQQNTNEKSLLELDQWDSLWSWF

SEQ ID NO: 72 - protein, 46 aa, Human Immunodeficiency virus 1
WQEWDRQISNISSTIYEEIQKAQVQQEQNEKKLLELDEWASIWNWL

SEQ ID NO: 73 - protein, 34 aa, Human Immunodeficiency virus 1
WQEWDRQISNISSTIYEEIQKAQVQQEQNEKKLL

SEQ ID NO: 74 - protein, 36 aa, Human Immunodeficiency virus 1
ISSTIYEEIQKAQVQQEQNEKKLLELDEWASIWNWL

SEQ ID NO: 75 - protein, 53 aa, Respiratory Syncytial Virus
GEPIINFYDPLVFPSDEFDASISQVHEKINQSLAFIRKSDELLHNVNAGKSTT

SEQ ID NO: 76 - protein, 56 aa, Human Parainfluenza Virus Type 3
YTPNDITLNNSVALDPIDISIELNKAKSDLEESKEWIRRSNQKLDSIGNWHQSSTT

SEQ ID NO: 77 - protein, 50 aa, Morbillivirus measles virus
PDAVYLHRIDLGPPISLERLDVGTNLNAIAKLEDAKELLESSDQILRSMK

SEQ ID NO: 78 - protein, 6 aa, Human Immunodeficiency virus 1
ELDKWA

(19) United States
(12) Patent Application Publication
(10) Pub. No.: US 2004/0132011 A1
(43) Pub. Date: Jul. 8, 2004

(54) Method for detecting viral inactivating agents

(75) Inventors: Graham P. Allaway, Darnestown, MD (US); Carl T. Wild, Gaithersburg, MD (US); Karl Salzwedel, Olney, MD (US)

Correspondence Address: STERNE, KESSLER, GOLDSTEIN & FOX PLLC, 1100 NEW YORK AVENUE, N.W., WASHINGTON, DC, 20005, US

(73) Assignee: Panacos Pharmaceuticals, Inc.

(21) Appl. No.: 10/685,801

(22) Filed: Oct. 16, 2003

Related U.S. Application Data
(60) Provisional application No. 60/418,341, filed on Oct. 16, 2002.

(51) Int. Cl. C12Q 1/70

(52) U.S. Cl. 435/5

(57) ABSTRACT

The invention is directed to methods for identifying compounds that decrease the ability of a virus, such as HIV-1, to infect previously uninfected cells by inducing conformational changes in viral envelope proteins, and the compounds discovered by such methods.

### CROSS-REFERENCE TO RELATED APPLICATIONS

This application claims priority benefit under 35 U.S.C. § 119(d)(e) to U.S. Provisional Application No. 60/418,341, filed Oct. 16, 2002, the entire contents of which are incorporated herein by reference.

### BACKGROUND OF THE INVENTION

1. Field of the Invention

The invention is directed to methods for identifying compounds that decrease the ability of a virus, such as HIV-1, to infect previously uninfected cells by inducing conformational changes in viral envelope proteins, and the compounds discovered by such methods.

2. Background Art

Enveloped viruses infect host cells via a series of events culminating in membrane fusion and viral entry into the host cell. Hoffman, L. R. et al., J. Virology 71:(11) 8808-8820 (1997). As such, membrane fusion is a potential target of research to prevent viral infection.

The HIV-1 envelope glycoprotein is a 160 kDa glycoprotein that is cleaved to form the transmembrane (TM) subunit, gp41, which is non-covalently attached to the surface (SU) subunit, gp120 (Allan J. S., et al., Science 228:1091-1094 (1985); Veronese F. D., et al., Science 229:1402-1405 (1985)). Recent efforts have led to a clearer understanding of the structural components of the HIV-1 envelope system. Such efforts include crystallographic analysis of significant portions of both gp120 and gp41 (Kwong, P. D., et al., Nature (London) 393:648-659 (1998); Chan, D. C., et al., Cell 89:263-273 (1997); Weissenhom, W., et al., Nature 387:426-430 (1997)).

The surface subunit has been characterized crystallographically as part of a multi-component complex consisting of the SU protein (the gp120 core absent the variable loops) bound to a soluble form of the cellular receptor CD4 (N-terminal domains 1 and 2 containing amino acid residues 1-181) and an antigen binding fragment of a neutralizing antibody (amino acid residues 1-213 of the light chain and 1-229 of the heavy chain of the 17b monoclonal antibody) which blocks chemokine receptor binding (Kwong, P. D., et al., Nature (London) 393:648-659 (1998)). Several envelope structures believed to exist only in the fusogenic form of gp120 were revealed by the crystallographic analysis including a conserved binding site for the chemokine receptor, a CD4-induced epitope and a cavity-laden CD4-gp120 interface. This supports earlier observations of CD4-induced changes in gp120 conformation.

The gp120/gp41 complex is believed to be present as a trimer on the virion surface where it mediates virus attachment and fusion. HIV-1 replication is initiated by the high affinity binding of gp120 to the cellular receptor CD4 and the expression of this receptor is a primary determinant of HIV-1 cellular tropism in vivo (Dalgleish, A. G., et al., Nature 312:763-767 (1984); Lifson, J. D., et al., Nature 323:725-728 (1986); Lifson, J. D., et al., Science 232:1123-1127 (1986); McDougal, J. S., et al., Science 231:382-385 (1986)). The gp120-binding site on CD4 has been localized to the CDR2 region of the N-terminal VI domain of this four-domain protein (Arthos, J., et al., Cell 5:469-481 (1989)). The CD4-binding site on gp120 maps to discontinuous regions of gp120 including the C2, C3 and C4 domains (Olshevsky, U., et al., Virol 64:5701-5707 (1990); Kwong, P. D., et al., Nature (London) 393:648-659 (1998)). Following attachment to CD4, the virus must interact with a “second” receptor such as a chemokine receptor in order to initiate the fusion process. Recently, researchers have identified the critical role of members of the chemokine receptor family in HIV entry (McDougal J. S., et al., Science 231:382-385 (1986); Feng Y., et al., Science 272:872-877 (1996); Alkhatib G., et al., Science 272:1955-1958 (1996); Doranz B. J., et al., Cell 85:1149-1158 (1996); Deng H., et al., Nature 381:661-666 (1996); Dragic T., et al., Nature 381:667-673 (1996); Choe H., et al., Cell 85:1135-1148 (1996); Dimitrov D. S., Nat. Med. 2:640-641 (1996); Broder, C. C. and Dimitrov, D. S., Pathobiology 64:171-179 (1996)). CCR5 is the chemokine receptor used by macrophage-tropic and many T-cell tropic primary HIV-1 isolates. Most T-cell line-adapted strains use CXCR4, while many T-cell tropic isolates are dual tropic, capable of using both CCR5 and CXCR4.

Binding of gp 120 to CD4 and a chemokine receptor initiates a series of conformational changes within the HIV envelope system (Eiden, L. E. and Lifson, J. D., Immunol. Today 13:201-206 (1992); Sattentau, Q. J. and Moore J. P., J. Exp. Med. 174:407-415 (1991); Allan J. S., et al., AIDS Res Hum Retroviruses 8:2011-2020 (1992); Clapham, P. R., et al., J. Virol. 66:3531-3537 (1992)). These changes occur in both the surface and transmembrane subunits and result in the formation of envelope structures which are necessary for virus entry. The functions of gp41 and gp120 appear to involve positioning the virus and cell membranes in close proximity thereby facilitating membrane fusion (Bosch M. L., et al., Science 244:694-697 (1989); Slepushkin, V. A. et al., AIDS Res Hum Retroviruses 8:9-18 (1992); Freed E. O. et al., Proc. Natl. Acad. Sci. USA 87:4650-4654 (1990)).

A good deal of structural information is available with respect to the HIV-1 transmembrane glycoprotein (gp41). This protein contains a number of well-characterized functional regions. See FIG. 1 For example, the N-terminal region consists of a glycine-rich sequence referred to as the fusion peptide which is believed to function by insertion into and disruption of the target cell membrane (Bosch, M. L., et al., Science 244:694-697 (1989); Slepushkin, V. A., et al., AIDS Res. Hum. Retrovirus 8:9-18 (1992); Freed, E. O., et al., Proc. Natl. Acad. Sci. USA 87:4650-4654 (1990); Moore, J. P., et al., “The HIV-cell Fusion Reaction,” in Viral Fusion Mechanism, Bentz, J., ed., CRC Press, Inc., Boca Raton, Fla.). Another region, characterized by the presence of disulfide linked cysteine residues, has been shown to be immunodominant and is suggested as a contact site for the surface (gp120) and transmembrane glycoproteins (Gnann, J. W., Jr., et al., J. Virol. 61:2639-2641 (1987); Norrby, E., et al., Nature 329:248-250 (1987); Xu, J. Y., et al., J. Virol. 65:4832-4838 (1991)). Other regions in the gp41 ectodomain have been associated with escape from neutralization (Klasse, P. J., et al., Virology 196:332-337 (1993); Thali, M., et al., J. Virol. 68:674-680 (1994); Stem, T. L., et al., J. Virol. 69:1860-1867 (1995)), immunosuppression (Cianciolo, G. J., et al., Immunol. Lett. 19:7-13 (1988); Ruegg, C. L., et al., J. Virol. 63:3257-3260 (1989)), and target cell binding (Qureshi, N. M., et al., AIDS 4:553-558 (1990); Ebenbichler, C. F., et al., AIDS 7:489-495 (1993); Henderson, L. A. and Qureshi, M. N., J. Biol. Chem. 268:15291-15297 (1993)).

Two regions of the ectodomain of gp41 have been shown to be critical to virus entry. Primary sequence analysis predicted that these regions (termed the N-helix (residues 558-595 of the HIV-1_{LAI} sequence) and C-helix (residues 643-678 of the HIV-1_{LAI} sequence) model α-helical secondary structure. Experimental efforts stemming from previous structural studies of synthetic peptide mimics established that the sequence analysis predictions were generally correct (Wild, C., et al., Proc. Natl. Acad. Sci. USA 89:10537-10541 (1992); Wild, C. T., et al., Proc. Natl. Acad. Sci. USA 91:9770-9774 (1994); Gallaher, W. R., et al., AIDS Res. Hum. Retroviruses 5:431-440 (1989); Delwart, E. L., et al., AIDS Res. Hum. Retroviruses 6:703-704 (1990)). Subsequent structural analysis determined that these regions of the transmembrane protein interact in a specific fashion to form a higher order structure characterized as a trimeric six-helix bundle (Chan, D. C., et al., Cell 89:263-273 (1997); Weissenhom, W., et al., Nature 387:426-430 (1997)). This trimeric structure consists of an interior parallel coiled-coil trimeric core (region one, N-helix) which associates with three identical α-helices (region two, C-helix) which pack in an oblique, antiparallel manner into the hydrophobic grooves on the surface of the coiled-coil trimer. This hydrophobic self-assembly domain is believed to constitute the core structure of gp41. See FIGS. 3A and 3B. It has been demonstrated that the N- and C-helical regions of the transmembrane protein are critical to HIV-1 entry. It has been proposed that the association of these two regions to form the six-helix bundle core structure occurs during the transition from a nonfusogenic to a fusogenic form of gp41, and that the formation of this core structure facilitates membrane fusion by bringing the viral and target cell surfaces into close proximity (Chan, D. C. and Kim, P. S., Cell 93:681-684 (1998); FIG. 1). If correct, the formation of the six-helix bundle is a key step in virus entry and factors which interfere with its formation could disrupt the entry event. A number of viruses share glycoprotein structures similar to the N- and C-helical regions of HIV transmembrane protein (Lambert et al., Proc. Nat. Acad. Sci. 93:2186-2191 (1996). See also, Published PCT Application No. WO96/19495.

All approved drugs for the treatment of human immunodeficiency virus (HIV) infection target viral reverse transcriptase (RT), protease activity, or viral fusion. Although certain combinations of these drugs have proven highly effective in suppressing virus replication, problems related to complicated dosing regimens and selection for resistant viral isolates necessitate the continued need for the development of additional therapies.

Mono- and bi-therapy for human immunodeficiency virus type 1 (HIV-1) infection are only transiently effective mainly due to virus drug resistance. To obtain a sustained benefit from antiviral therapy, current guidelines recommend at least triple-drug combinations, or the so-called highly active antiretroviral therapy (HAART). Despite these advances, there are still problems with the currently available drug regimens. Many of the drugs exhibit severe toxicities or require complicated dosing schedules that reduce compliance and limit efficacy. Resistant strains of HIV usually appear over extended periods of time even on HAART regimens.

For these and other reasons there is a continuing need for the development of additional anti-HIV drugs. Ideally these would target different stages in the viral life cycle, (adding to the armamentarium for combination therapy), exhibit minimal toxicity, and have low manufacturing costs. Small molecule inhibitors of HIV entry could aid significantly in addressing these problems.

### BRIEF SUMMARY OF THE INVENTION

The present invention is directed to a method of screening for compounds that decrease the ability of a virus to infect previously uninfected cells. The present invention provides methods of screening for compounds that induce conformational changes in viral envelope proteins that result in loss of function by envelope structures necessary for virus entry into permissive cells. The screening methods involve identifying compounds that selectively induce function-impairing changes in the conformation of one or more structures necessary for virus entry found in cell-surfaced-expressed viral envelope proteins and probing for such changes. This can be accomplished as described herein.

A method for identifying compounds that decrease the ability of a virus to infect previously uninfected cells, comprising:

provide a cell, virion, pseudovirion, membrane vesicle, lipid bilayer, or liposome expressing or bearing viral envelope protein or glycoprotein or fragment thereof,

contact said cell, virion, pseudovirion, membrane vesicle, lipid bilayer, or liposome with a candidate compound; and

measure the ability of said candidate compound to induce conformational changes in viral envelope glycoprotein or fragments thereof by determining binding of an antibody, antibody fragment or peptide to said viral envelope glycoprotein or fragments thereof.

The method described above, wherein said virus is a retrovirus, such as HIV.

The method described above, wherein the ability of said candidate compound to induce conformational changes in viral envelope glycoprotein or fragments thereof, is measured by determining binding of a peptide to the induced conformations.

The method described above, wherein the ability of said candidate compound to induce conformational changes in viral envelope glycoprotein or fragments thereof, is measured by determining binding of an antibody or antibody fragment to the induced conformations.

The method described above, wherein said antibody is either a monoclonal or polyclonal antibody.

The method described above, wherein the antibody or antibody fragment used to measure the ability of said candidate compound to induce conformational changes in viral envelope glycoprotein or fragments thereof, comprise single chain, light chain, heavy chain, CDR, F(ab′)₂, Fab, Fab′, Fv, sFv or dsFv or any combination thereof.

The method described above, wherein the antibody, antibody fragment or peptide are labeled with a labeling agent. The labeling agent may be an enzyme, fluorescent substance, chemiluminescent substance, horseradish peroxidase, alkaline phosphatase, biotin, avidin, electron dense substance, or radioisotope, or combinations thereof.

The method described above, wherein said contact of said cell, virion, pseudovirion, membrane vesicle, lipid bilayer, or liposome with a candidate compound, optionally occurs in the presence of cellular receptors. The cellular receptors may include, among others, CD4 (soluble or membrane bound), fragments of CD4, chemokine receptors, CCR5, CXCR4 or combinations thereof.

A specific embodiment of the invention is directed to a method for determining compounds which induce changes in the conformation of critical gp41 structures necessary for virus entry and therefore block HIV entry. The gp41 six-helix bundle which forms in response to CD4/gp120 binding constitutes one such critical entry structure. Previous studies have demonstrated that soluble CD4 can bind to gp120 on the surface of HIV-1 virions and cause the loss of gp120 from the surface, resulting in viral inactivation. Similarly, studies have shown that sCD4 interacts with gp120/gp41 on HIV infected cells resulting in conformational changes in gp41 (six-helix bundle formation). In the present invention small molecule inhibitors of virus entry are identified by their ability to interact with either gp120 or gp41 in the absence of cellular receptors resulting in the formation of the six-helix bundle structure in gp41 and inactivating virus.

Compounds that induce conformation changes in the assays of the current invention may act at any of the several steps leading to, or associated with, the conformation changes in the viral envelope glycoproteins that result in membrane fusion. For example, such compounds may induce the interaction between the envelope glycoprotein and its receptors which initiates conformation changes in the envelope glycoproteins (e.g. in the case of HIV-1, the interaction between gp120 and CD4 or the CCR5 or CXCR4 chemokine receptors). Alternatively, they may directly induce the formation of fusion active structures, e.g, by causing the association of the alpha helical domains of the transmembrane protein that are part of one of these structures (e.g. in the case of HIV-1, by inducing the association of the N- and C-helical domains, elicit six helix bundle formation). The assays are also capable of discovering inducing mechanisms of other steps in the process that are as yet not fully elucidated.

Further, certain compounds discovered by the method of the present invention cause the loss of gp120 from the virus surface or interact with gp120 at the CD4 binding site or the chemokine receptor binding site or elsewhere to induce conformational changes in gp41. Compounds of this invention, therefore can function similarly to CD4, binding gp120. In some cases these molecules may be mimics of the action of CD4 or chemokine receptors. They can also interact directly with gp41 to induce changes in the structure of gp41. Antibodies specific for the gp41 six-helix bundle can be used to determine the ability of candidate compounds to induce its formation.

Several methods can be used to detect binding of antibodies in these assays, including, immunoprecipitation analysis, flow cytometry, fluorescence microscopy, or fluorometry, enzyme assays, radiolabeling or chemiluminescence techniques.

The methods of the present invention can be applied to other viruses where a transmembrane protein or glycoprotein forms structures and complexes that are involved for virus entry, including but not limited to, HIV-2, HTLV-I, HTLV-II, respiratory syncytial virus (RSV), human influenza viruses, parainfluenza virus type 3 (HPIV-3), measles virus, hepatitis B virus (HBV) and hepatitis C virus (HCV) or other viruses, such as retroviruses or enveloped viruses. Enveloped viruses include viruses with a capsid surrounded by a lipid bilayer.

The invention is also directed to novel compounds identified by these methods, which can be small molecules, peptides, proteins, antibodies and antibody fragments. These compounds decrease the ability of a virus to infect previously uninfected cells. The compounds of this invention can be used to treat humans infected with HIV-1 or the other viruses. The invention also includes compounds identified by the method described above in suitable pharmaceutical compositions. These compounds can also be used to inactivate viruses in body fluids e.g., blood or blood components used for therapeutic purposes.

### BRIEF DESCRIPTION OF THE DRAWINGS/FIGURES

FIG. 1 illustrates the postulated role of gp41 in mediating virus entry. In the native state, the HIV-1 envelope complex exists in a nonfusogenic form. Following CD4 (and in some cases chemokine) binding, a pre-hairpin intermediate forms. At this point, the transmembrane protein, gp41, is in an extended conformation and the N- and C-helical domains have yet to associate. This intermediate proceeds to form the six-helix bundle (hairpin intermediate). Formation of the bundle serves to facilitate virus-target cell fusion by drawing the viral and cellular membranes close together. In the presence of a triggering compound, the pre-hairpin intermediate (extended conformation) is formed. Following the formation of the six-helix bundle (hairpin intermediate) structure, the virus is incapable of fusing to a permissive cell.

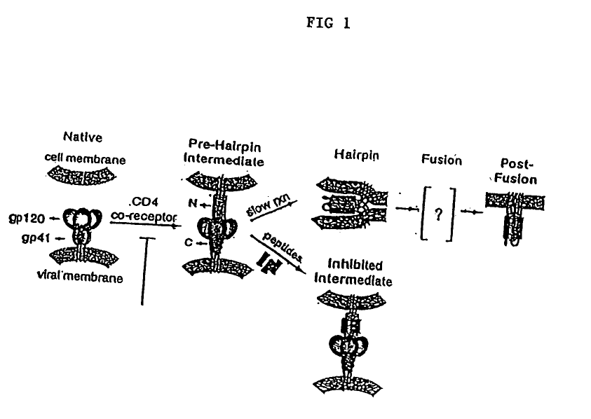

FIG. 2 is a schematic representation of the structural and antigenic regions of HIV-1 gp41. This figure also depicts conformational changes that occur in these regions when an antibody binds to gp41.

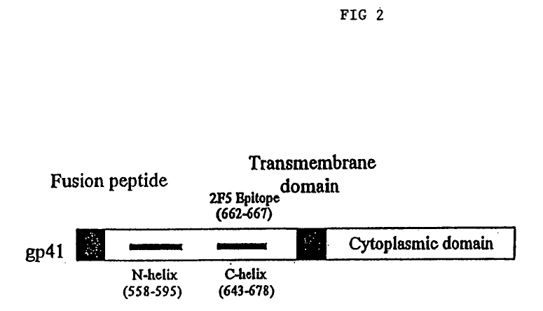

FIGS. 3A and 3B are schematic representations of the interaction of the N- and C-helical domains of gp41 to form the six-helix bundle structure. Both top and side views are shown. The interior of the bundle represents the N-helical coiled-coil. The exterior components represent the C-helical domain.

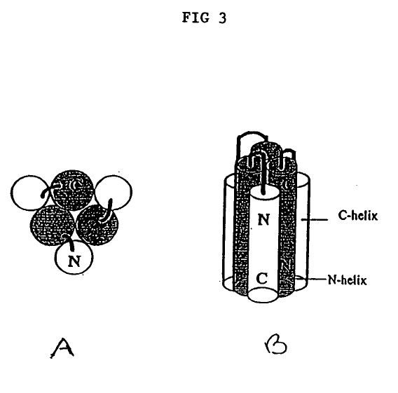

FIG. 4 is a schematic representation of gp41 intermediate structures formed during virus entry. Fusion intermediate I forms immediately following receptor binding and shows the ectodomain in an extended form. Fusion intermediate II shows gp41 following core structure formation. Triggering these conformational intermediates in the absence of CD4 renders a virus incapable of fusion when in contact with a permissive cell.

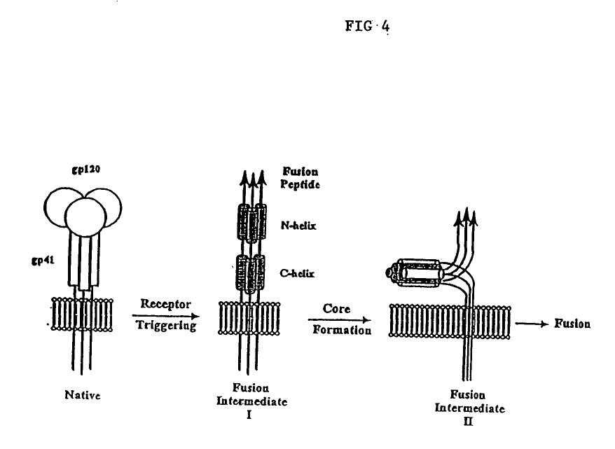

FIGS. 5A and 5B are a schematic representation of the structural and antigenic regions of HIV-1 gp41. These figures also show the conformational changes that these regions typically undergo upon binding of an antibody specific for the gp41 core structure.

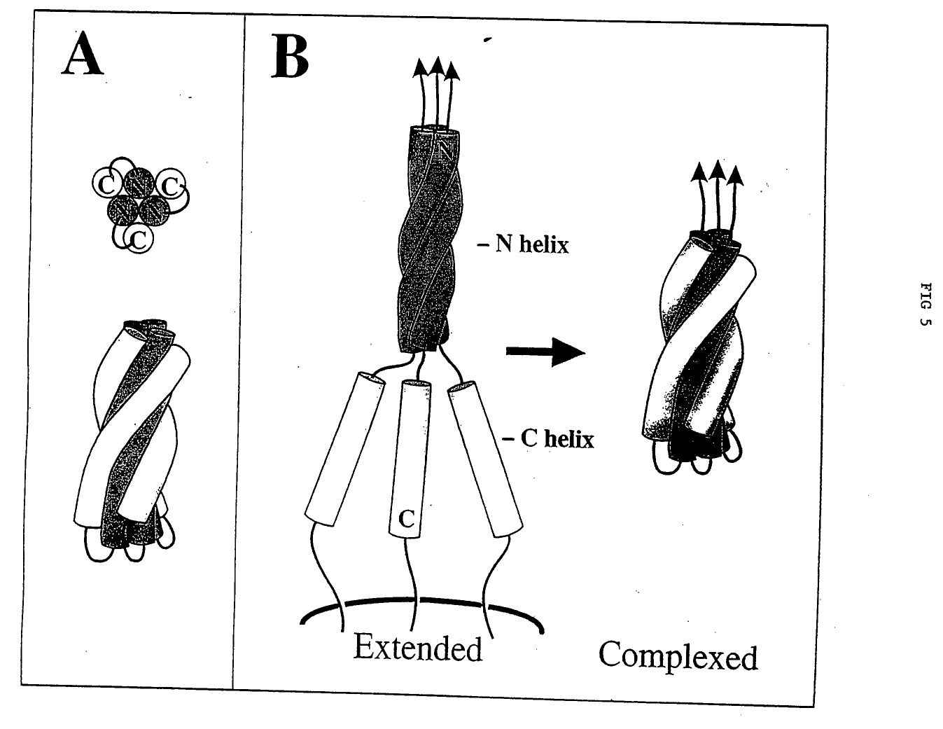

### DETAILED DESCRIPTION OF THE INVENTION

The present invention is directed to a method of screening for compounds that decrease the ability of a virus to infect previously uninfected cells. The present invention provides methods of screening for compounds that induce conformational changes in viral envelope proteins that result in loss of function by envelope structures necessary for virus entry into permissive cells. The screening methods involve identifying compounds that selectively induce function-impairing changes in the conformation of one or more structures necessary for virus entry found in cell-surfaced-expressed viral envelope proteins and probing for such changes. This can be accomplished as described herein.

In a first aspect, the present invention is directed to a screening assay for inhibitory compounds which involves determining the ability of a candidate compound to induce conformational changes in viral envelope protein or glycoprotein or fragments thereof, such that the conformational changes render the cell, virion, pseudovirion, membrane vesicle, lipid bilayer or liposome expressing or bearing envelope protein or glycoprotein no longer fusogenic. In particular, the method comprises:

A method for identifying compounds that decrease the ability of a virus to infect previously uninfected cells, comprising:

provide a cell, virion, pseudovirion, membrane vesicle, lipid bilayer, or liposome expressing or bearing viral envelope protein or glycoprotein or fragment thereof,

contact said cell, virion, pseudovirion, membrane vesicle, lipid bilayer, or liposome with a candidate compound; and

measure the ability of said candidate compound to induce conformational changes in viral envelope glycoprotein or fragments thereof by determining binding of an antibody, antibody fragment or peptide to said viral envelope glycoprotein or fragments thereof.

The method described above, wherein said virus is a retrovirus, such as HIv.

The method described above, wherein the ability of said candidate compound to induce conformational changes in viral envelope glycoprotein or fragments thereof, is measured by determining binding of a peptide to the induced conformations.

The method described above, wherein the ability of said candidate compound to induce conformational changes in viral envelope glycoprotein or fragments thereof, is measured by determining binding of an antibody or antibody fragment to the induced conformations.

The method described above, wherein the antibody or antibody fragment used to measure the ability of said candidate compound to induce conformational changes in viral envelope glycoprotein or fragments thereof, comprise single chain, light chain, heavy chain, CDR, F(ab′)₂, Fab, Fab′, Fv, sFv or dsFv or any combination thereof.

The method described above, wherein the antibody, antibody fragment or peptide are labeled with a labeling agent. The labeling agent may be an enzyme, fluorescent substance, chemiluminescent substance, horseradish peroxidase, alkaline phosphatase, biotin, avidin, electron dense substance, or radioisotope, or combinations thereof.

The method described above, wherein said contact of said cell, virion, pseudovirion, membrane vesicle, lipid bilayer, or liposome with a candidate compound optionally occurs in the presence of cellular receptors. The cellular receptors may include CD4 (soluble or membrane bound), fragments of CD4, chemokine receptors, CCR5 or CXCR4, or combinations thereof.

Preferably, providing a cell, virion, pseudovirion, membrane vesicle, lipid bilayer or liposome expressing or bearing viral envelope protein or glycoprotein, and contacting said cell, virion, pseudovirion, membrane vesicle, lipid bilayer or liposome with a candidate compound comprises incubating the cell, virion, pseudovirion, membrane vesicle, lipid bilayer or liposome expressing or bearing viral envelope protein or glycoprotein or fragments thereof and the candidate compound for about 10 minutes to about 120 minutes, more preferably about 30 to about 90 minutes. Useful concentration ranges of candidate compound include from about 0.1 μg/mL to about 100 μg/mL. Useful concentration ranges of viral envelope protein or glycoprotein or fragments thereof vary widely and may depend upon the manner upon which the viral envelope protein or glycoprotein or fragments thereof are provided as discussed below.

The ability of a candidate compound to induce conformational changes can be measured by antibody binding to the induced conformations. The detection antibodies are either monoclonal or polyclonal antibodies. Useful antibodies include antibodies raised against combinations of peptides, recombinant proteins, proteins, and protein fragments that accurately model envelope structures necessary for virus entry. Methods of generating these antibodies and determining their binding are discussed below.

Detection of induced conformational changes is carried out by incubating the mixture of proteins, glycoproteins, or fragments thereof in the association with a cell, virion, pseudovirion, membrane vesicle, lipid bilayer or lipsome with a candidate compound, with specific antibodies to determine whether the amount of antibody binding to an induced conformation necessary for viral entry or fusion is increased or decreased due to the presence of the candidate compound. An increase in antibody binding to an induced conformation in the presence of a candidate compound compared to a standard value, indicates a positive result.

Alternatively, the ability of a candidate compound to induce a change in conformation can be measured by antibody binding to viral envelope protein or glycoprotein or fragments thereof, as it exists prior to contact with a candidate compound. Methods of generating these antibodies and determining their binding are discussed below. The detection antibodies that bind to epitopes present in the viral envelope protein or glycoprotein or fragments thereof should bind to epitopes present only prior to the induction of entry-related conformational changes. Therefore, in this aspect, antibody binding indicates a negative result.

In one embodiment, the measuring of the ability of a candidate compound to induce a change in conformation is performed by:

adding one or more optionally detectably-labeled antibodies that preferentially bind an epitope that is present in an induced conformation or structure required for virus entry; and

measuring the amount of antibody binding.

A positive result using such measuring method is observed by a detecting a greater amount of bound antibody compared to a standard value.

In another embodiment, the ability of a candidate compound to induce a change in conformation is determined by:

adding one or more optionally detectably-labeled antibodies that preferentially bind an epitope that is only present on a viral envelope protein or glycoprotein prior to receptor induction; and

measuring the amount of antibody binding.

A positive result using such measuring method is observed by a detecting a lesser amount of bound antibody compared to a standard value.

Useful viral envelope proteins or glycoproteins are those proteins and/or glycoproteins that have one or more domains that participate in the entry event of a virus into a virus permissive cell. For instance, HIV-1 includes the envelope glycoproteins gp 120/gp41. The envelope glycoprotein gp41 includes an N-helical domain and C-helical domain that participate in forming structures required for HIV fusion and entry into HIV-permissive cells (for example, lymphocytes). Other viruses, such as RSV, parainfluenza virus type 3 (HPIV-3), measles virus, and influenza virus include functionally similar envelope glycoprotein primary and secondary structure which form structures and conformations that mediate viral fusion and entry. The protein or glycoprotein or fragments thereof, are associated with an appropriate cell, virion, pseoudovirion, membrane vesicle, lipid bilayer or liposome.

Another aspect of the present invention, is directed to a method for identifying compounds with the ability to induce the formation of one or more critical gp41 structures or conformations necessary for entry, and thereby block HIV entry. The gp41 six-helix bundle structure which forms in response to CD4/gp120 binding constitutes one such critical entry structure. Antibodies specific for the six-helix bundle structure are used to determine the ability of small molecules to induce its formation. An increase in antibody binding to the six-helix bundle structure after incubation with a candidate compound compared to a standard value, indicates a positive result.

Thus, the present invention also provides a method for identifying compounds that decrease the ability of HIV-1 to infect previously uninfected cells, comprising:

provide HIV-1 envelope glycoproteins gp120/gp41 or fragments thereof in association with a cell, virion, pseoudovirion, membrane vesicle, lipid bilayer or liposome;

contact said HIV-1 envelope glycoproteins gp120/gp41 or fragments thereof with a candidate compound; and

measure the ability of said candidate compound to induce changes that result in the formation of entry structures in gp41.

In that method of the present invention, the measuring is performed by detecting changes in the conformation of gp41 using poly- and/or monoclonal sera raised against a mixture of peptides or recombinant proteins mimicking the six-helix bundle structure. Such polyclonal sera are generated by immunizing animals with a 1:1 mixture of the P15 and P16 peptides. Alternatively, the ability of a candidate compound to induce conformational changes in gp41 is detected by using monoclonal antibodies, including T26, 17b, 48d, 8F101 or A32, or mixtures thereof. (See, e.g. Earl et al, J Virol 1997 April; 71(4):2674-84), and NC-1 (Jiang et al, J Virol 1998 December; 72(12):10213-7).

Additional antibodies useful for detecting conformational changes in gp120 include 17b (Sullivan et al, J Virol 1998 June; 72(6):4694-703), 48d (Thali et al, J Virol 1993 July; 67(7):3978-88), 8F101 (DeVico et al, Virology 1995 Aug. 20; 211(2):583-8) and A32 (Wyatt et al, J Virol 1995 September; 69(9):5723-33). Candidate compounds that induce the formation of an entry structure, such as a six-helix bundle, would cause an increase in binding of these antibodies.

Alternatively, the effect a candidate compound has on HIV-1 envelope glycoproteins gp120/gp41 is measured by detecting the presence of gp120. Antibody binding to gp120 indicates that the candidate compound has not induced a change in conformation that causes the loss of gp120 from the surface of a cell.

The present invention also pertains to viral envelope proteins or glycoproteins of HIV-1, HIV-2, HTLV-I, HTLV-II, respiratory syncytial virus (RSV), parainfluenza virus type 3 (HPIV-3), human influenza viruses, measles virus, or other enveloped viruses. Enveloped viruses have a capsid surrounded by a lipid bilayer. The present invention also pertains to viral envelope proteins or glycoproteins of hepatitis B virus (HBV) or hepatitis C virus (HCV).

For purposes of the invention, a viral envelope protein or glycoprotein can be in association with a lipid bilayer in a number of different ways, so long as the viral envelope protein or glycoprotein exists in one or more conformations similar to a conformation that the protein or glycoprotein exists in its native environment. It is important that the protein or glycoprotein or fragments thereof be in an environment which allows the protein or glycoprotein or fragments thereof to form functional entry structures and conformations as defined herein.

Cells expressing the envelope glycoprotein or fragment thereof are cells infected with a recombinant vaccinia virus expressing the HIV-1 envelope protein or fragment thereof. In another embodiment, the cells expressing the envelope glycoprotein or fragment thereof are cells transformed with a vector expressing the HIV-1 envelope protein or fragment thereof. In another embodiment, the cells expressing the envelope glycoprotein or fragment thereof are infected with a replication defective viral particle or pseudovirion bearing at least one envelope protein or fragment thereof from at least one laboratory-adapted or primary virus infected cells.

Useful lipid bilayer systems include cells, virions, pseudovirions or other appropriate membrane vesicles or liposomes expressing or bearing either a viral envelope protein or glycoprotein or fragments thereof. The envelope viral protein or glycoprotein will typically have one or more membrane-associating domains and one or more transmembrane domains. Examples of useful lipid bilayer systems in the present invention include: cells transfected such that they surface express membrane associated envelope protein or glycoprotein, cells infected with replication defective viral particles and surface expressed membrane associated envelope protein or glycoprotein, inactivated virus particles, and pseudovirions.

The method of the present invention can be applied to viruses where a transmembrane protein or glycoprotein forms structures, conformations, and complexes that are involved with virus entry, including but not limited to, HIV-1, HIV-2, HTLV-I, HTLV-II, respiratory syncytial virus (RSV), parainfluenza virus type 3 (HPIV-3), human influenza viruses, measles virus, hepatitis B virus (HBV) or hepatitis C virus (HCV) or other enveloped viruses.

For purposes of the present invention, a “virus-permissive cell” is a cell into which a particular virus typically can enter and infect.

Useful virus permissive cells, or insoluble or soluble receptors from said virus permissive cells are dictated by the particular virus, and the host cells which are permissive to fusion and entry of the particular virus. For example, for HIV-1, permissive cells include lymphocytes. For RSV, HEp2 cells are useful permissive cells. For measles virus, Vero cells are useful permissive cells. For HIPV-3, HEp2 cells are useful permissive cells.

The phrase “induce conformational changes” as employed herein is the induction of structures and conformational intermediates necessary for viral fusion and entry into permissive cells. The changes in conformation render a cell, virion, pseudovirion, membrane vesicle, lipid bilayer or lipsome expressing or bearing envelope protein, glycoprotein or fragments thereof, no longer fusogenic by the induction of such conformational structures and intermediates away from a permissive cell. In particular, by inducing conformational changes in the absence of CD4, viral fusion intermediates form away from a permissive cell. This renders the virus incapable of fusion when proximal to a permissive cell.

Several methods can be used to detect binding of the antibodies in the methods of the present invention, including immunoprecipitation analysis, flow cytometry, fluorescence microscopy, or fluorometry. In addition, enzyme assays, radiolabelling such as, radioimmunoassay (RIA), and chemiluminescense techniques can be employed.

The antibodies are optionally labeled with a detectable label. Suitable labels are known in the art and include enzyme labels, such as, alkaline phosphatase, horseradish peroxidase, and glucose oxidase, and radioisotopes, such as iodine (¹²⁵I, ¹²¹I), carbon (¹⁴C), sulfur (³⁵S), tritium (³H), indium (¹¹²In), and technetium (^{99m}Tc), and fluorescent labels, such as europium, fluorescein and rhodamine. Alternatively, the antibodies can be derivatized with a moiety that is recognized by a separately-added label, for example, biotin. Techniques for chemically modifying antibodies with these labels are well-known in the art.

The measuring step optionally further comprises comparing the amount of antibody binding to a standard value. Antibody binding can be measured and expressed in a number of ways that are known to one of ordinary skill in the art, including enzyme assays, immunoprecipitation analysis, flow cytometry, fluorescence microscopy, or fluorometry, radiolabeling or chemiluminescence techniques.

Useful reagents in the present invention include non-infectious HIV-1 particles (an example being 8E5/LAV virus (Folks, T. M., et al., J. Exp. Med. 164:280-290 (1986); Lightfoote, M. M., et al., J. Virol. 60:771-775 (1986); Gendelman, H. E., et al., Virology 160:323-329(1987))) or pseudovirions bearing the envelope glycoprotein or fragment thereof from at least one laboratory-adapted or primary HIV-1 isolate or virus infected cell (Haddrick, M., et al., J. Virol. Methods 61:89-93 (1996); Yamshchikov, G. V., et al., Virology 21:50-58 (1995)).

The 8E5/LAV cell line produces an intact virion expressing functional envelope in a non-replicating system. A soluble form or fragment thereof of the primary HIV-1 receptor, CD4, is added (sCD4).

In another alternative embodiment, cells expressing at least one viral envelope protein, e.g., cells infected with a recombinant vaccinia virus expressing the HIV-1 envelope protein or fragment thereof (Earl, P. L., et al., J. Virol. 65:31-41 (1991); Rencher, S. D., et al., Vaccine 5:265-272 (1997); Katz, E. and Moss, B., AIDS Res. Hum. Retroviruses 13:1497-1500 (1997)), can be used.

The invention includes the novel compounds detected in these assays that may include but are not limited to small molecules, peptides, antibodies and antibody fragments, or derivatives thereof. The small molecules detected in these assays have a molecular weight of less than 500, less than 1000 or less than 2000.

The invention, in particular, includes compounds that cause the loss of gp120 from the surface of a virus cell, decreasing the ability of said virus to infect previously uninfected cells.

The invention, in particular, includes compounds that change the conformation of gp41 by inducing the formation of the six-helix bundle, decreasing the ability of said virus to infect previously uninfected cells.

The invention, in particular, includes compounds that decrease the ability of HIV-1, HIV-2, HTLV-I, HTLV-II, respiratory syncytial virus (RSV), parainfluenza virus type 3 (HPIV-3), Newcastle disease virus, human influenza viruses, measles virus, hepatitis B virus (HBV) or hepatitis C virus (HCV) or other enveloped viruses, to infect previously uninfected cells by inducing the formation of necessary entry structures.

These inhibitors can be used to treat humans infected with HIV-1 or the other viruses, or used to decrease infection by HIV-1 or the other viruses. The invention also includes the inhibitors in suitable pharmaceutical compositions. These antiviral compounds can also be used to inactivate viruses in body fluids e.g. blood or blood components used for therapeutic purposes.

Antibodies to CD4 Induced Epitopes

The peptides and polypeptides useful in the present invention are preferably provided in an isolated form. By “isolated polypeptide” is intended a polypeptide removed from its native environment. Thus, a polypeptide produced and/or contained within a recombinant host cell is considered isolated for purposes of the present invention. Also intended as an “isolated polypeptide” are polypeptides that have been purified, partially or substantially, from a recombinant host cell or from a native source. For example, a recombinantly produced polypeptide can be substantially purified by the one-step method described in Smith and Johnson, Gene 67:31-40 (1988). Alternatively, peptides can be synthesized using well-known peptide synthesis techniques.

In one aspect of the invention antibodies are raised by administering to a mammal a peptide or polypeptide comprising an amino acid sequence that is capable of forming a stable coiled-coil solution structure corresponding to or mimicking the heptad repeat region of gp41 which is located in the N-helical domain as defined herein. Peptides, or multimers thereof, that comprise amino acid sequences which correspond to or mimic solution conformation of the N-helical heptad repeat region of gp41 can be employed. The N-helical heptad repeat region of gp41 includes 4 or more heptad repeats. Preferably, the peptides comprise about 28 to 55 amino acids of the heptad repeat region of the extracellular domain of HIV gp41 (N-helical domain, (SEQ. ID NO:1)), or multimers thereof. The peptides can be administered as a small peptide, or conjugated to a larger carrier protein such as keyhole limpet hemocyanin (KLH), ovalbumin, bovine serum albumin (BSA) or tetanus toxoid. Peptides forming a stable coiled-coil solution structure corresponding to or mimicking the heptad repeat region of gp41 can be employed to form either polyclonal or monoclonal antibodies. To determine whether a particular peptide or multimer will possess a stable trimeric coiled-coil solution structure corresponding to or mimicking the heptad repeat region of gp41, the peptide can be tested according to the methods described in Wild, C., et al., Proc. Natl. Acad. Sci. USA 89:10537-10541 (1992), fully incorporated by reference herein.

Shown below is the sequence for residues of the HIV-1_{LAI} gp41 protein that form the N-helical domain of the protein:

(SEQ. ID NO:1)

ARQLLSGIVQQQNNLLRAIEAQQHLLQLTVWGIKQLQARILAVERYLK
DQQLLGI

Two examples of useful peptides include the peptide P-17, which has the formula, from amino terminus to carboxy terminus, of:

NH₂—NNLLRAIEAQQHLLQLTVWGIKQLQARILAVERYLKDQ-COOH (SEQ ID NO:2);

and the peptide P-15, which has the formula, from amino terminus to carboxy terminus, of:

NH₂—SGIVQQQNNLLRAIEAQQHLLQLTVWGIKQLQARIL-COOH (SEQ ID NO:3).

These peptides are optionally coupled to a larger carrier protein, or optionally include a terminal protecting group at the N- and/or C-termini. Useful peptides further include peptides corresponding to P-17 or P-15 that include one or more, preferably 1 to 10 conservative substitutions, as described below. A number of useful N-helical region peptides are described herein.

Antibodies can also be raised by administering to a mammal a peptide or polypeptide comprising an amino acid sequence that corresponds to, or mimics, the transmembrane-proximal amphipathic α-helical segment of gp41 (C-helical domain, or a portion thereof. Useful peptides or polypeptides include an amino acid sequence that is capable of forming a six helix bundle when mixed with a peptide corresponding to the heptad repeat region of gp41, such as the peptide P-17. Peptides can be tested for the ability to form a six helix bundle employing the system and conditions described in Chan, D. C., et al, Cell 89:263-273 (1997); Lu, M., et al., Nature Struct. Biol. 2:1075-1082 (1995), fully incorporated by reference herein.

Shown below is the amino acid sequence for residues of the HIV-1 LA gp41 protein that form the C-helical domain of the protein:

(SEQ ID NO:4)

WNNMTWMEWDREINNYTSLIHSLIEESQNQQEKNEQELLELDKWASL
WNWFNITNW

Preferred peptides or multimers thereof, that can be employed in this aspect of the invention comprise about 6 or more amino acids, preferably about 24-56 amino acids, of the extracellular C-helical domain of HIV gp41. The peptides can be administered as a small peptide, or conjugated to a larger carrier protein such as keyhole limpet hemocyanin (KLH), ovalbumin, bovine serum albumin (BSA) or tetanus toxoid. This transmembrane-proximal amphipathic α-helical segment is exemplified by the peptides P-16 and P-18, described below. Peptides or polypeptides comprising amino acid sequences that correspond to, or mimic, the transmembrane-proximal amphipathic α-helical segment of gp41, or a portion thereof, can be employed to form either polyclonal or monoclonal antibodies.

Examples of useful peptides for this aspect of the invention include the peptide P-18 which corresponds to a portion of the transmembrane protein gp41 from the HIV-1_{LAI} isolate, and has the 36 amino acid sequence (reading from amino to carboxy terminus):

NH₂—YTSLIHSLIEESQNQQEKNEQELLELDKWASLWNWF—COOH (SEQ ID NO:5);

and the peptide P-16, which has the following amino acid sequence (reading from amino to carboxy terminus):

NH₂—WMEWDREINNYTSLIHSLIEESQNQQEKNEQELL—COOH (SEQ ID NO:6)

These peptides are optionally coupled to a larger carrier protein. Useful peptides further include peptides corresponding to P-18 or P-16 that include one or more, preferably 1 to 10 conservative substitutions, as described below. In addition to the full-length P-18, 36-mer and the full length P-16, the peptides of this aspect of the invention may include truncations of the P-18 and P-16, as long as the truncations are capable of forming a six helix bundle when mixed with P-17 or P-15.

Antibodies can also be raised by administering to a mammal one or more peptides or polypeptides which comprise amino acid sequences that are capable of forming solution stable structures that correspond to, or mimic, the gp41 six helix bundle. This bundle forms in gp41 by the interaction of the distal regions of the transmembrane protein, the heptad repeat region and the amphipathic α-helical region segment roughly corresponding to the N-helical domain and C-helical domain. The bundle structures that form in native virus are the result of a trimeric interaction between three copies each of the heptad repeat region and the transmembrane-proximal amphipathic α-helical segment. In the compositions useful in the present invention, peptide regions interact with one another to form a six helix bundle. Useful are mixtures of peptides and polypeptides, including multimeric and conjugate structures, wherein said structures form a stable helical solution structure.

Mixtures of (a) one or more peptides that comprise an amino acid sequence that corresponds to, or mimics, a stable coiled coil heptad repeat region of gp41; and (b) one or more peptides that comprise a region that corresponds to, or mimics, the transmembrane-proximal amphipathic α-helical segment of gp41 are contemplated. In addition to physical mixtures, and conventional cross-linking, the peptides (a) and (b) can be conjugated together via suitable linking groups, preferably a peptide residue having at least 2, preferably 2 to 25, amino acid residues. Preferred linking groups are formed from combinations of glycine and serine, or combinations of glycine and cysteine when further oxidative cross-linking is envisioned.

Exemplary embodiments include raising antibodies to physical mixtures of P-17 and P-18, P-15 and P-16, P-17 and P-16 or P-15 and P-18.

Antibodies can also be raised by administering to a mammal a composition including one or more novel peptides and proteins, herein referred to as conjugates, that mimic transmembrane protein entry structures. These conjugates are formed from peptides and proteins that comprise:

(a) one or more amino acid sequences of 28 or more amino acids that are capable of forming a stable coiled-coil solution structure corresponding to or mimicking the heptad repeat region of gp41; and

(b) one or more amino acid sequences that correspond to, or mimic, an amino acid sequence of the transmembrane-proximal amphipathic α-helical segment of gp41;

wherein

said one or more sequences (a) and (b) are alternately linked to one another via a peptide bond (amide linkage) or by an amino acid linking sequence consisting of about 2 to about 25 amino acids. These peptides and proteins are preferably recombinantly produced.

These conjugates preferably fold and assemble into a structure corresponding to, or mimicking, a gp41 entry structure. Examples of the novel constructs or conjugates that can be formed include (reading from N-terminus to C-terminus):

(1) three tandem repeating units consisting of P-17-linker-P-18 (P-17-linker-P-18-linker-P-17-linker-P-18-linker-P-17-linker-P-18),

(2) P-17-linker-P-18-linker-P-17,

(3) P-18-linker-P-17-linker-P-18,

(4) P-18-linker-P-17,

(5) three tandem repeating units consisting of P-15-linker-P-16 (P-15-linker-P-16-linker-P-15-linker-P-16-linker-P-15-linker-P-16),

(6) P-15-linker-P-16-linker-P-15,

(7) P-16-linker-P-15-linker-P-16,

(8) P-16-linker-P-15; and

(9) P-15-linker-P-16;

wherein each linker is an amino acid sequence, which may be the same or different, of from about 2 to about 25, preferably 2 to about 16 amino acid residues. Preferred amino acid residues include glycine and serine, for example (GGGGS)ₓ, (SEQ ID NO:7) wherein x is 1, 2, 3, 4, or 5, or glycine and cysteine, for example (GGC)Y, where y is 1, 2, 3, 4 or 5. In any of the described constructs, P-15 and P-17 are interchangeable and P-16 and P-18 are interchangeable.

The phrase “entry structure” as employed herein, refers to particular molecular conformations or structures that occur or are exposed following interaction of HIV with the cell surface during viral entry, and the role of particular amino acid sequences and molecular conformation or structures in viral entry.

The term “HIV” as used herein refers to all strains and isolates of human immunodeficiency virus type 1. Certain constructs employed in the invention were based upon HIV-I gp41, and the numbering of amino acids in HIV proteins and fragments thereof given herein is with respect to the HIV-1_{LAI} isolate. However, it is to be understood, that while HIV-1 viral infection and the effects of the present invention on such HIV-1 infection are being used herein as a model system, the entry mechanism that is being targeted is relevant to all strains and isolates of HIV-1. Hence the invention is directed to “comprehensive screening” methods.

The phrase “heptad repeat” or “heptad repeat region” as employed herein, refers to a common protein motif having a 4-3 repeat of amino acids, leucine and/or isoleucine often found at the 1 and 4 positions, and is often associated with alphα-helical secondary structure. The “heptad repeat” can be represented by the following sequence:

-(AA₁-AA2-AA3-AA4-AA5-AA6-AA7)-

where AA₁ and AA4 are each one of leucine or isoleucine; while AA2, AA3, AA5, AA6, and AA7 can be any amino acid. See, Wild, C., et al., Proc. Natl. Acad. Sci. USA 89:10537-10541 (1992).

Peptides are defined herein as organic compounds comprising two or more amino acids covalently joined by peptide bonds. Peptides may be referred to with respect to the number of constituent amino acids, i.e., a dipeptide contains two amino acid residues, a tripeptide contains three, etc. Peptides containing ten or fewer amino acids may be referred to as oligopeptides, while those with more than ten amino acid residues are polypeptides.

The complete gp41 amino acid sequence (HIV-1 Group M: Subtype B Isolate: LAI, N to C termini) is:

| | |
|---|---|
| AVGIGALFLGFLGAAGSTMGARSMTLTVQARQLLSGIVQQQNNLLRAIEA | (SEQ ID NO:8) |
| QQHLLQLTVWGIKQLQARILAVERYLKDQQLLGIWGCSGKLICTTAVPWN | |
| ASWSNKSLEQIWNNMTWMEWDREINNYTSLIHSLIEESQNQQEK | |
| NEQELLELDKWASLWNWFNITNWLWYIKIFIMIVGGLVGLRIVFAVLSIV | |
| NRVRQGYSPLSFQTHLP-TPRG-PDRPEGIEEEGGERDRDRSIRLVNGSL | |
| ALIWDDLRSLCLFSYHRLRDLLLIVTRIVELLGRRGWEALKYWW | |
| NLLQYWSQELKNSAVSLLNATAIAVAEGTDRVIEVVQGACRAIRHIPRRIR | |
| QGLERILL. | |

The N-terminal helical region of gp41 is:

| | |
|---|---|
| ARQLLSGIVQQQNNLLRAIEAQQHLLQLTVWGIKQLQARILAVERYLKDQ | (SEQ ID NO:1) |
| QLLGI | |

Shown below is the sequence for residues 558-595 (SEQ ID NO:7) of the HIV-1_{LAI} gp41 protein in the N-helical domain of the protein. The a and d subscripts denote the 4-3 positions of the heptad repeat.

| N N L L R A I E A Q Q H L L Q L T V W G I K Q L Q A R I L A V E R Y L K D Q | (SEQ ID NO:2) |
|---|---|
| d a d a d a d a d a | |
| 571 578 585 | |

The C-terminal helical region of gp41 is:

| | |
|---|---|
| WNNMTWMEWDREINNYTSLIHSLIEESQNQQEKNEQELLELDKWASL | (SEQ ID NO:4) |
| WNWFNITNW | |

Shown below is the amino acid sequence for residues 643-678 of the HIV-1_{LAI} gp41 protein in the C-helical domain of the protein.

| Y T S L I H S L I E E S Q N QQ E K N E Q E L L E L D K W A S L W N W F | (SEQ ID NO:5) |
|---|---|
| d a d a d a d a d a | |
| 647 654 661 | |

Peptides modeling the N and C-helical domains of HIV-1 gp41 can be constructed from multiple strains of HIV, and can include amino acid deletions, insertions and substitutions that do not destroy the ability of the resulting peptides to elicit antibodies against gp41 entry structures and conformations when employed alone or in combination with other peptides of the invention.

The effect of such changes on the ability of peptides modeling the N-helical region of gp41 to elicit the desired antibody response can be determined spectrophotometrically. Deletions, insertions and substitutions within the primary sequence of N-helical peptides which do not alter the ability of the peptide to form α-helical secondary structure as measured by circular dichroism (Wild, C. et al., PNAS 89:10537-10541 (1992) are considered compatible with their use in the invention.

When modeled as a peptide, the C-helical region of gp41 is not structured. However, when mixed with the N-peptide, the C-peptide does take on a α-helical secondary structure as part of the six-helical core complex. The structure forms in vitro on mixing N- and C-helical peptides and can be characterized spectrophotometrically (Lu, M., et al., Nat. Struct. Biol. 2:1075-1082 (1995)). The initial determination of the effect of primary sequence deletions, insertions and substitutions on C-helix structure may be performed by analyzing the ability of the variant C-peptides to interact with a structured form of the N-peptide to form the six-helix bundle. C-peptides which interact to forms this structure are considered compatible with their use in the invention. This analysis may be carried out using circular dichroism.

Examples of N-helical Domain Peptide Sequences (All sequences are listed from N-terminus to C-terminus.) from different HIV strains include, but are not limited to the following peptides:

| | |
|---|---|
| HIV-1 Group M: Subtype B Isolate: LAI | |
| ARQLLSGIVQQQNNLLRAIEAQQHLLQLTVWGIKQLQARILAVERYLK | (SEQ ID NO:1) |
| DQQLLGI | |
| SGIVQQQNNLLRAIEAQQHLLQLTVWGIKQLQARILAVERYLKDQ | (SEQ ID NO:9) |
| P15 SGIVQQQNNLLRAIEAQQHLLQLTVWGIKQLQARIL | (SEQ ID NO:3) |
| P-17 NNLLRAIEAQQHLLQLTVWGIKQLQARILAVERYLKDQ | (SEQ ID NO:2) |
| Subtype B Isolate: ADA | |
| SGIVQQQNNLLRAIEAQQHLLQLTVWGIKQLQARVLALERYLRDQ | (SEQ ID NO:10) |
| SGIVQQQNNLLRAIEAQQHLLQLTVWGIKQLQARVL | (SEQ ID NO:11) |
| NNLLRAIEAQQHLLQLTVWGIKQLQARVLALERYLRDQ | (SEQ ID NO:12) |
| Subtype B Isolate: JRFL | |
| SGIVQQQNNLLRAIEAQQRMLQLTVWGIKQLQARVLAVERYLGDQ | (SEQ ID NO:13) |
| SGIVQQQNNLLRAIEAQQRMLQLTVWGIKQLQARVL | (SEQ ID NO:14) |
| NNLLRAIEAQQRMLQLTVWGIKQLQARVLAVERYLGDQ | (SEQ ID NO:15) |
| Subtype B Isolate: 89.6 | |
| SGIVQQQNNLLRAIEAQQHMLQLTVWGIKQLQARVLALERYLRDQ | (SEQ ID NO:16) |
| SGIVQQQNNLLRAIEAQQHMLQLTVWGIKQLQARVL | (SEQ ID NO:17) |
| NNLLRAIEAQQHMLQLTVWGIKQLQARVLALERYLRDQ | (SEQ ID NO:18) |
| Subtype C Isolate: BU910812 | |
| SGIVQQQSNLLRAIEAQQHMLQLTVWGIKQLQARVLAIERYLRDQ | (SEQ ID NO:19) |
| SGIVQQQSNLLRAIEAQQHMLQLTVWGIKQLQARVL | (SEQ ID NO:20) |
| SNLLRAIEAQQHMLQLTVWGIKQLQARVLAIERYLRDQ | (SEQ ID NO:21) |
| Subtype D Isolate: 92UG024D | |
| SGIVQQQNNLLRAIEAQQHLLQLTVWGIKQLQARVLAVESYLKDQ | (SEQ ID NO:22) |
| SGIVQQQNNLLRAIEAQQHLLQLTVWGIKQLQARVL | (SEQ ID NO:11) |
| NNLLRAIEAQQHLLQLTVWGIKQLQARVLAVESYLKDQ | (SEQ ID NO:23) |
| Subtype F Isolate: BZ163A | |
| SGIVQQQSNLLRAIEAQQHLLQLTVWGIKQLQARVLAVERYLQDQ | (SEQ ID NO:24) |
| SGIVQQQSNLLRAIEAQQHLLQLTVWGIKQLQARVL | (SEQ ID NO:25) |
| SNLLRAIEAQQHLLQLTVWGIKQLQARVLAVERYLQDQ | (SEQ ID NO:26) |
| Subtype G Isolate: FI.HH8793 | |
| SGIVQQQSNLLRAIEAQQHLLQLTVWGIKQLQARVLALERYLRDQ | (SEQ ID NO:27) |
| SGIVQQQSNLLRAIEAQQHLLQLTVWGIKQLQARVL | (SEQ ID NO:25) |
| SNLLRAIEAQQHLLQLTVWGIKQLQARVLALERYLRDQ | (SEQ ID NO:28) |
| Subtype H Isolate: BE.VI997 | |
| SGIVQQQSNLLRAIQAQQHMLQLTVWGVKQLQARVLAVERYLKDQ | (SEQ ID NO:29) |
| SGIVQQQSNLLRAIQAQQHMLQLTVWGVKQLQARVL | (SEQ ID NO:30) |
| SNLLRAIQAQQHMLQLTVWGVKQLQARVLAVERYLKDQ | (SEQ ID NO:31) |
| Subtype J Isolate: SE.SE92809 | |
| SGIVQQQSNLLKAIEAQQHLLKLTVWGIKQLQARVLAVERYLKDQ | (SEQ ID NO:32) |
| SGIVQQQSNLLKAIEAQQHLLKLTVWGIKQLQARVL | (SEQ ID NO:33) |
| SNLLKAIEAQQHLLKLTVWGIKQLQARVLAVERYLKDQ | (SEQ ID NO:34) |
| Group N Isolate: CM.YBF30 | |
| SGIVQQQNILLRAIEAQQHLLQLSIWGIKQLQAKVLAIERYLRDQ | (SEQ ID NO:35) |
| SGIVQQQNILLRAIEAQQHLLQLSIWGIKQLQAKVL | (SEQ ID NO:36) |
| NILLRAIEAQQHLLQLSIWGIKQLQAKVLAIERYLRDQ | (SEQ ID NO:37) |
| Group O Isolate: CM.ANT70C | |
| KGIVQQQDNLLRAIQAQQQLLRLSxWGIRQLRARLLALETLLQNQ | (SEQ ID NO:38) |
| KGIVQQQDNLLRAIQAQQQLLRLSxWGIRQLRARL | (SEQ ID NO:39) |
| DNLLRAIQAQQQLLRLSxWGIRQLRARLLALETLLQNQ | (SEQ ID NO:40) |

Examples of C-helical Domain Peptide Sequences (All sequences are listed from N-terminus to C-terminus.) from different HIV strains include, but are not limited to the following peptides:

| | |
|---|---|
| HIV-1 Group M: Subtype B Isolate: LAI | |
| WNNMTWMEWDREINNYTSLIHSLIEESQNQQEKNEQELLELDKWASL | (SEQ ID NO:4) |
| WNWFNITNW | |
| WMEWDREINNYTSLIHSLIEESQNQQEKNEQELLELDKWASLWNWF | (SEQ ID NO:41) |
| P16 WMEWDREINNYTSLIHSLIEESQNQQEKNEQELL | (SEQ ID NO:6) |
| P-18 YTSLIHSLIEESQNQQEKNEQELLELDKWASLWNWF | (SEQ ID NO:5) |
| Subtype B Isolate: ADA | |
| WMEWEREIENYTGLIYTLIEESQNQQEKNEQDLLALDKWASLWNWF | (SEQ ID NO:42) |
| WMEWEREIENYTGLIYTLIEESQNQQEKNEQDLL | (SEQ ID NO:43) |
| YTGLIYTLIEESQNQQEKNEQDLLALDKWASLWNWF | (SEQ ID NO:44) |
| Subtype B Isolate: JRFL | |
| WMEWEREIDNYTSEIYTLIEESQNQQEKNEQELLELDKWASLWNWF | (SEQ ID NO:45) |
| WMEWEREIDNYTSEIYTLIEESQNQQEKNEQELL | (SEQ ID NO:46) |
| YTSEIYTLIEESQNQQEKNEQELLELDKWASLWNWF | (SEQ ID NO:47) |
| Subtype B Isolate: 89.6 | |
| WMEWEREIDNYTDYIYDLLEKSQTQQEKNEKELLELDKWASLWNWF | (SEQ ID NO:48) |
| WMEWEREIDNYTDYIYDLLEKSQTQQEKNEKELL | (SEQ ID NO:49) |
| YTDYIYDLLEKSQTQQEKNEKELLELDKWASLWNWF | (SEQ ID NO:50) |
| Subtype C Isolate: BU910812 | |
| WIQWDREISNYTGIIYRLLEESQNQQENNEKDLLALDKWQNLWSWF | (SEQ ID NO:51) |
| WIQWDREISNYTGIIYRLLEESQNQQENNEKDLL | (SEQ ID NO:52) |
| YTGIIYRLLEESQNQQENNEKDLLALDKWQNLWSWF | (SEQ ID NO:53) |
| Subtype D Isolate: 92UG024D | |
| WMEWEREISNYTGLIYDLIEESQIQQEKNEKDLLELDKWASLWNWF | (SEQ ID NO:54) |
| WMEWEREISNYTGLIYDLIEESQIQQEKNEKDLL | (SEQ ID NO:55) |
| YTGLIYDLIEESQIQQEKNEKDLLELDKWASLWNWF | (SEQ ID NO:56) |
| Subtype F Isolate: BZ163A | |
| WMEWQKEISNYSNEVYRLIEKSQNQQEKNEQGLLALDKWASLWNWF | (SEQ ID NO:57) |
| WMEWQKEISNYSNEVYRLIEKSQNQQEKNEQGLL | (SEQ ID NO:58) |
| YSNEVYRLIEKSQNQQEKNEQGLLALDKWASLWNWF | (SEQ ID NO:59) |
| Subtype G Isolate: FI.HH8793 | |
| WIQWDREISNYTQQIYSLIEESQNQQEKNEQDLLALDNWASLWTWF | (SEQ ID NO:60) |
| WIQWDREISNYTQQIYSLIEESQNQQEKNEQDLL | (SEQ ID NO:61) |
| YTQQIYSLIEESQNQQEKNEQDLLALDNWASLWTWF | (SEQ ID NO:62) |
| Subtype H Isolate: BE.VI997 | |
| WMEWDRQIDNYTEVIYRLLELSQTQQEQNEQDLLALDKWDSLWNWF | (SEQ ID NO:63) |
| WMEWDRQIDNYTEVIYRLLELSQTQQEQNEQDLL | (SEQ ID NO:64) |
| YTEVIYRLLELSQTQQEQNEQDLLALDKWDSLWNWF | (SEQ ID NO:65) |
| Subtype J Isolate: SE.SE92809 | |
| WIQWEREINNYTGIIYSLIEEAQNQQENNEKDLLALDKWTNLWNWFN | (SEQ ID NO:66) |
| WIQWEREINNYTGIIYSLIEEAQNQQENNEKDLL | (SEQ ID NO:67) |
| YTGIIYSLIEEAQNQQENNEKDLLALDKWTNLWNWFN | (SEQ ID NO:68) |
| Group N Isolate: CM.YBF30 | |
| WQQWDEKVRNYSGVIFGLIEQAQEQQNTNEKSLLELDQWDSLWSWF | (SEQ ID NO:69) |
| WQQWDEKVRNYSGVIFGLIEQAQEQQNTNEKSLL | (SEQ ID NO:70) |
| YSGVIFGLIEQAQEQQNTNEKSLLELDQWDSLWSWF | (SEQ ID NO:71) |
| Group O Isolate: CM.ANT70C | |
| WQEWDRQISNISSTIYEEIQKAQVQQEQNEKKLLELDEWASIWNWL | (SEQ ID NO:72) |
| WQEWDRQISNISSTIYEEIQKAQVQQEQNEKKLL | (SEQ ID NO:73) |
| ISSTIYEEIQKAQVQQEQNEKKLLELDEWASIWNWL | (SEQ ID NO:74) |

The peptides and conjugates may be acylated at the NH₂ terminus, and may be amidated at the COOH terminus.

Useful peptides from fusion proteins from other viruses that function during entry include the following peptides.

For RSV:

GEP NFYDPLVFPSDEFDASISQVHEKINQSLAFIRKSDELLHNVNAGKSTT (SEQ ID NO:75)

For HPIV3:

(SEQ ID NO:76)

YTPNDITLNNSVALDPIDISIELNKAKSDLEESKEWIRRSNQKLDSIGNW
HQSSTT

For measles virus:

PDAVYLHRIDLGPPISLERLDVGTNLNAIAKLEDAKELLESSDQILRSMK (SEQ ID NO:77)

Additional useful peptides are described in PCT Published Application No. Published PCT Application No. WO96/19495, and U.S. Pat. Nos. 6,020,459, 6,017,536, 6,013,263, 6,008,044 and 6,015,881, all of which are fully incorporated by reference herein. The peptides and conjugates may be acylated at the NH₂ terminus, and may be amidated at the COOH terminus. Mixtures and conjugates of the appropriate N-helical and C-helical peptides can be employed to generate antibodies to entry conformations and structures. The peptides can be employed alone to generate antibodies to the appropriate viral membrane protein or glycoprotein.

The peptides and conjugates may include conservative amino acid substitutions. Conserved amino acid substitutions consist of replacing one or more amino acids of the peptide sequence with amino acids of similar charge, size, and/or hydrophobicity characteristics, such as, for example, a glutamic acid (E) to aspartic acid (D) amino acid substitution. When only conserved substitutions are made, the resulting peptide is functionally equivalent to the peptide from which it is derived.

Peptide sequences defined herein are represented by one-letter symbols for amino acid residues as follows:

| | |
|---|---|
| A | alanine |
| R | arginine |
| N | asparagine |
| D | aspartic acid |
| C | cysteine |
| Q | glutamine |
| E | glutamic acid |
| G | glycine |
| H | histidine |
| I | isoleucine |
| L | leucine |
| K | lysine |
| M | methionine |
| F | phenylalamine |
| P | proline |
| S | serine |
| T | threonine |
| W | tryptophan |
| Y | tyrosine |
| V | valine |

The peptides and conjugates useful in the invention may include amino acid insertions which consist of single amino acid residues or stretches of residues ranging from 2 to 15 amino acids in length. One or more insertions may be introduced into the peptide, peptide fragment, analog and/or homolog.

The peptides and conjugates useful in the invention may include amino acid deletions of the full length peptide, analog, and/or homolog. Such deletions consist of the removal of one or more amino acids from the full-length peptide sequence, with the lower limit length of the resulting peptide sequence being 4 to 6 amino acids. Such deletions may involve a single contiguous portion or greater than one discrete portion of the peptide sequences.

Listed below are other useful antibodies:

the 2F5 monoclonal antibody which is the only broadly neutralizing antibody targeting gp41. This antibody maps to the linear amino acid sequence Glu-Leu-Asp-Lys-Trp-Ala (ELDKWA)(SEQ ID NO:78) in the ectodomain of obtainable from AIDS gp41 an epitope which is conserved in 72% of HIV-I isolates; and

monoclonal antibody, NC-1, which has been shown to bind the six-helix bundle in sCD4-activated gp41. NC-1, was generated and cloned from a mouse immunized with a mixture of peptides modeling the N— and C-helical domains of gp41. NC—I binds specifically to both the α-helical core domain and the oligomeric forms of gp41. This conformational-dependent reactivity is dramatically reduced by point mutations within the N-terminal coiled-coil region of gp41 which impede formation of the gp41 core. NC—I binds to the surfaces of HIV-1-infected cells only in the presence of soluble CD4.

Immunogen Preparation

Immunogens can be prepared by several different routes. The constructs can be generated from synthetic peptides. This involves preparing each sequence as a peptide monomer followed by post-synthetic modifications to generate the appropriate oligomeric structures. The peptides are synthesized by standard solid-phase methodology. To generate a trimeric coiled-coil structure, the P-15 or P-17 peptide monomer is solubilized under conditions which favor oligomerization. These conditions include a 20 mM phosphate buffer, pH 4.5 and a peptide concentration of 100 μM (Wild, C., et al., Proc. Natl. Acad. Sci. USA 89:10537-10541 (1992)). The structure which forms under these conditions can be optionally stabilized by chemical crosslinking, for example using glutaraldehyde.

Alternatively, a protocol which makes use of intermolecular disulfide bond formation to stabilize the trimeric coiled-coil structure can be employed in order to avoid any disruptive effect the cross-linking process might have on the structural components of this construct. This approach uses the oxidation of appropriately positioned cysteine residues within the peptide sequence to stabilize the oligomeric structure. This requires the addition of a short linker sequence to the N terminus of the P-17 peptide. The trimeric coiled-coil structure which is formed by this approach will be stabilized by the interaction of the cysteine residues. The trimer is separated from higher order oligomeric forms, as well as residual monomer, by size exclusion chromatography and characterized by analytical ultracentrifugation. These covalently stabilized coiled-coil oligomers serve as the core structure for preparation of a six helix bundle.

To accomplish preparation of a six helix bundle, an excess of P-18 peptide or P-16 peptide is added to the N-helical coiled-coil trimer. After incubation the reaction mixture is optionally subjected to a cross-linking procedure to stabilize the higher order products of the specific association of these two peptides. The desired material is isolated by size exclusion chromatography and can be characterized by analytical ultracentrifugation. The immunogen corresponding only to the P-18 or P-16 peptide requires no specific post-synthetic modifications. Using this approach, three separate target constructs are generated rapidly and in large amounts.

Another method for preparing target immunogens involves the use of a bacterial expression vector to generate recombinant gp41 fragments. The use of an expression vector to produce the peptides and polypeptides capable of forming the entry-structure-containing immunogens of the present invention adds a level of versatility to immunogen preparation.

New and modified forms of the antigenic targets are contemplated as the structural determinants of HIV-1 entry are better understood. The recombinant approach readily accommodates these changes. Also, this method of preparation allows for the ready modification of the various constructs (i.e. the addition of T- or B-cell epitopes to the recombinant gp41 fragments to increase immunogenicity). Finally, these recombinant constructs can be employed as a tool to provide valuable insights into additional structural components which form and function in gp41 during the process of virus entry.

To generate a six helix bundle structure, several combinations of the heptad repeat (for example, P-17 or P-15) region and the membrane proximal amphipathic α-helical (for example, P-16 or P-18) segment of gp41 are separated by a flexible linker of amino acid residues. For example, (GGGGS)ₓ (SEQ ID NO:7) where x is 1, 2 or 3 can be encoded into the vector. This is accomplished by standard PCR methods. The (GGGGS)ₓ (SEQ ID NO:7) linker motif is encoded by a synthetic oligonucleotide which is ligated between the P-17 and P-18 encoding regions of the expression vector.

All constructions are characterized by multiple restriction enzyme digests and sequencing. The success of this approach to attain multicomponent interactions has been recently demonstrated (Huang, B., et al., J. Immunol. 158:216-225 (1997)).

Following expression, the recombinant gp41 fragments are isolated as inclusion bodies, cleaved from the leader sequence by cyanogen bromide, and separated from the leader by-product by size exclusion chromatography step (SUPERDEX 75). This protocol has been successfully used in the purification of large quantities of a modified form of the P-17 peptide (Calderone, T. L., et al., J. Mol. Biol. 262:407-412 (1996)). Recombinant constructs (2) and (3) are mixed in equal molar quantities under non-denaturing conditions to generate a six-helix bundle structure. Constructs (1) and (4) will fold either intra- or intermolecularly to generate the same or similar structures. The desired product is purified by size exclusion chromatography on a SUPERDEX 75 FPLC column and characterized by molecular weight using a Beckman Model XL-A analytical ultracentrifuge.

Antibody Generation and Characterization

Generation and characterization of the antibodies against novel gp41 epitopes constitutes the second aspect of the invention. The experimental sera and monoclonal antibodies generated against the target immunogens are subjected to thorough biophysical and biological evaluation.

For the production of antibodies to entry structures, various host animals may be immunized by injection with a differentially expressed gene protein, or a portion thereof. Such host animals may include but are not limited to rabbits, mice, and rats, to name but a few. Various adjuvants may be used to increase the immunological response, depending on the host species, including but not limited to Freund's (complete and incomplete), mineral gels such as aluminum hydroxide, surface active substances such as lysolecithin, pluronic polyols, polyanions, peptides, oil emulsions, keyhole limpet hemocyanin, dinitrophenol, and potentially useful human adjuvants such as BCG (bacille Calmette-Guerin) and Corynebacterium parvum.

Polyclonal antibodies are heterogeneous populations of antibody molecules derived from the sera of animals immunized with an antigen, such as a peptide or mixtures or conjugates thereof as described above. For the production of polyclonal antibodies, host animals such as those described herein, may be immunized by injection with one or more peptides or recombinant proteins optionally supplemented with adjuvants.

Monoclonal antibodies, which are homogeneous populations of antibodies to a particular antigen, may be obtained by any technique which provides for the production of antibody molecules by continuous cell lines in culture. These include, but are not limited to the hybridoma technique of Kohler and Milstein, (Nature 256:495-497 (1975); and U.S. Pat. No. 4,376,110), the human B-cell hybridoma technique (Kosbor et al., Immunology Today 4:72 (1983); Cole et al., Proc. Natl. Acad. Sci. USA 80:2026-2030 (1983)), and the EBV-hybridoma technique (Cole et al., Monoclonal Antibodies And Cancer Therapy, Alan R. Liss, Inc., pp. 77-96 (1985)). Such antibodies may be of any immunoglobulin class including IgG, IgM, IgE, IgA, IgD and any subclass thereof. The hybridoma producing the mAb of this invention may be cultivated in vitro or in vivo. Production of high titers of mAbs in vivo makes this the presently preferred method of production.

Antibodies can be generated following established protocols. All small animal work (immunizations, bleeds, and hybridoma production) is carried out by standard methods known to those of skill in the art. A first set of immunogens consists of the peptide constructs P-15 or P-17 (capable of forming trimeric coiled-coil multimers, optionally stabilized by chemical cross-linking or oxidation), P-16 or P-18, and the P-17/P-18 mixture or P-15/P-16 mixture (wherein the peptides are optionally chemically or oxidatively cross-linked). In one set of experiments, the immunogens are conjugated to a carrier such as KLH.

Balb-c mice are immunized with each of these constructs. Mice can receive 100 μg of antigen conjugated to KLH. Following the initial immunization the animals receive a 100 μg boost on day 14 followed by 50 μg boosts on days 30 and 45. Bleeds occur two weeks following the final boost. Mice are also immunized with the recombinant constructs following the same outline as that for the peptide immunogens.

Alternative immunization approaches include the use of a recombinant adenovirus vector expressing all or part of the HIV-1 envelope glycoprotein gp120/gp41 as the primary immunogen followed by booster immunizations with the gp41 peptides, proteins or other constructs.

Samples can be screened by ELISA to characterize antibody binding. The antigenpanel includes all experimental immunogens. Animals with sera samples which test positive for binding to one or more experimental immunogens are candidates for use in MAb production. Following this initial screen, one animal representing each experimental immunogen is selected for monoclonal antibody production.

Hybridoma supernatants are screened by ELISA, against structured and non-structured peptides and recombinants. Samples that are ELISA negative or weakly positive are further characterized for IgG. If IgG is present the material is screened in the biophysical and biological assays. Strongly positive samples are screened for their ability to neutralize viral envelope.

Antibodies are characterized in detail for their ability to bind HIV envelope under various conditions. For detection of antibody binding to native envelope, immunoprecipitations on Env-expressing cells and virions, both intact and lysed are performed using non-ionic detergents (Furata, R A et al., Nat. Struct. Biol. 5(4):276-279 (1997); White, J. M. and I. A. Wilson, J. Cell Biol. 105:2887-2894 (1987); Kemble, G. W., et al., J. Virol. 66:4940-4950 (1992)). Antibody binding to cell lysates and intact virions are also assayed in an ELISA format. Flow cytometry experiments are performed to determine binding to envelope expressing cells. Cross-competition experiments using other mapped Mabs, human sera, and peptides can also be performed. To characterize “triggers” to the conformational change, antibody binding to virus in the presence and absence of both sCD4 and target cells can be compared (White, J. M. and I. A. Wilson, J. Cell Biol. 105:2887-2894 (1987); Kemble, G. W., et al., J. Virol. 66:4940-4950 (1992)). Because the gp41 regions are highly conserved, epitope exposure using several different envelopes can be compared to discern possible differences in structure between primary, lab-adapted and genetically diverse virus isolates.

Binding of peptide anti-sera to viral envelope is analyzed using immunoblot and immunoprecipitation (IP) assays. The results from these assays indicate that certain of the peptides and recombinant gp41 fragments accurately model envelope entry determinants and structures. The outcome of the Western blot studies should roughly parallel the results from the ELISA assays with antisera raised against the more stable structured immunogens exhibiting the strongest binding to viral envelope determinants. In the lysate immunoprecipitation assay, polyclonal sera generated against the P 15, P 17, and P15/P 17 mixed peptides as well as rgp41 precipitate the viral transmembrane protein.

To further determine the ability of these immunogens to generate antibodies against gp41 entry structures a series of surface immunoprecipitation assays are carried out. These experiments allow characterization of antibody binding to cell-surface expressed envelope prior to and post receptor triggering. This assay format allows the study of epitopes found in both non-flisogenic and fusogenic envelope. In these experiments CD4 in both soluble and cell-expressed forms is utilized as a trigger for gp41 activation. The results indicate that both an N-helical peptide, the mixture of N- and C-helical peptides, and rgp41 generate antibodies against gp41 entry structures. The greatly enhanced binding by antisera raised against the six-helix bundle post CD4 triggering is consistent with the proposed role of this gp41 determinant in virus entry.

ELISA Assay

Nunc Immulon 2 HB plates are coated with 1 μg/well of peptide. Approximately, 100 μl of sample at desired dilution are added in duplicate and allowed to incubate for 2 hrs at 37° C. Hybridoma supernatants are tested neat while polyclonal sera are assayed at an initial concentration of 1:100 followed by 4-fold serial dilutions. Following incubation, samples are removed and plates are washed with PBS+0.05% Tween-20, and 100 pl/well of diluted phosphatase-labeled secondary antibody (Sigma) is added. The secondary antibody-conjugate is diluted in blocking buffer to a final concentration of 1:1500 and added. Following incubation at room temperature, plates are washed and substrate (Sigma fast p-nitrophenyl phosphate) is added. Following development, plates are read at 405 nm.

Western Blot Analysis

Commercial HIV-1 western blot strips are pre-wet with wash buffer (PBS+0.05% Tween-20). Samples are diluted in buffer (PBS, 0.05% Tween-20, 5% evaporated milk) to a final concentration of 1:5 for hybridoma supernatants and 1:200 for polyclonal sera and added to the strips. Following incubation (2 hrs with rocking), the strips are washed (3×5 min intervals) with wash buffer. Peroxidase-labeled secondary antibody (Kirkagard & Perry Laboratories) is added at a concentration of 1:5000 and incubated with rocking for 1 h. Strips are washed again as described previously and TMB substrate is added. Color development is stopped by the addition of water.

Lysate Immunoprecipitation Assay

Hybridoma supernatants or immunosera are incubated overnight at 4° C. in 200 μl PBS containing 4.2 μl of HIV-1 IIIB cell lysate. The lysate is prepared from acute infection of the H9 cell line. Immune complexes are precipitated by the addition of protein A and G Agarose, washed and analyzed by 10% SDS-PAGE (NOVEX), transferred to nitrocellulose and immunoblotted with anti-gp41 monoclonal antibody Chessie 8 (obtained from NIH AIDS Research and Reference Reagent Program), and detected by chemiluminescence (Amersham) and autoradiography.

Surface Immunoprecipitation Assay

Envelope expressing cells are prepared by acute infection of human 293T cells or other permissive cell line. U87 cells expressing CD4 with and without CXCR4 chemokine receptor are provided by D. R. Littman (New York University, New York, N.Y.). Surface Immunoprecipitation: Five days following infection, 5×10⁶ Env-expressing cells are incubated 1 h at desired temperature in 0.5 ml Dulbecco's Modified Eagle media (DMEM) in the presence or absence of soluble CD4 (Intracell Inc.) (final concentration 4 μM) or appropriate target cells (5×10⁶ cells in 0.5 ml media). 2 μl of immunosera or hybridoma supernatant is added and allowed to incubate for an additional hour. Cells are washed twice with phosphate buffered saline (PBS) and lysed with 200 μl of lysis buffer (1% Triton X-100, 150 mM NaCl, 50 mM Tris-HCl pH 7.4). The clarified supernatants are incubated 1 h at 4° C. with a mix of 12.5 μM protein A-Agarose/12.5 μM of protein G-Agarose (GIBCO BRL) followed by washing with lysis buffer (3×). Immunoprecipitated complexes are analyzed by 10% SDS-PAGE (NOVEX), transferred to nitrocellulose, and immunoblotted with anti-gp41 monoclonal antibody Chessie 8 (obtained from NIH AIDS Research and Reference Reagent Program), and detected by chemiluminescence (Amersham) and autoradiography.

Immunoprecipitation Studies

The panel of antibodies are tested by surface immunoprecipitation analysis for ability to bind HxB2 gp41 following the interaction of envelope expressing cells with sCD4 or cells expressing various receptor and co-receptor combinations. The surface expressed forms of CD4 and second receptor are furnished by the U87 cell line which has been engineered to selectively express CD4 only, CD4 plus CXCR4, and CD4 plus CCR5. In each case, incubations are performed at 37° C. for various periods of time (initially 5 minutes, 1, 4 and 12 hours as described below), then cooled to 4° C. to limit any further changes while immunoprecipitation is carried out. Immunoprecipitation is performed as described above.

Preparation of Envelope Expressing Cells

Envelope expressing cells are prepared by infection of U87 cells expressing CD4 and appropriate chemokine receptor or other permissive cell lines with the desired primary virus isolate at high multiplicity of infection (MOI). The level of envelope expression at a given MOI for each virus isolate is determined by the immunoblot procedure described previously. The MOI for each HIV isolate is adjusted to give similar levels of envelope expression in each case. The surface immunoprecipitation assay is carried out as described above.

### EXAMPLE 1

### Formation of Antibodies

Monoclonal antibodies against the gp41 six-helix bundle are prepared by standard methods. The immunogen used consists of a physical mixture of synthetic peptides modeling the N- and C-helical domains of an envelope protein or glycoprotein that function during the viral entry event. The immunogen consists of a physical mixture of synthetic peptides modeling the N- and C-helical gp41 domains.

| | | |
|---|---|---|
| N peptide: | S G I V Q Q Q N N L L R A I E A Q Q H L L Q L T V W G I K Q L Q A R I L. | (SEQ ID NO:3) |
| C peptide: | W M E W D R E I N N Y T S L I H S L I E E S Q N Q Q E K N E Q E L L | (SEQ ID NO:6) |

Four balb-c mice are immunized with this mixed construct. Following the initial immunization (100 μg) the animals receive a 100 μg boost on day 14 followed by 50 μg boosts on days 30 and 45. Bleeds occur two weeks following the final boost. The polyclonal sera generated by the immunization of experimental animals are screened by ELISA to characterize binding. Sera samples testing negative for binding by ELISA are abandoned. Animals with sera samples which test positive for binding to the experimental immunogen are candidates for use in monoclonal antibody (MAb) production. Following this initial screen, at least one animal is selected for MAb production. The criteria for this selection is based upon envelope binding patterns against the cognate immunogen. Hybridoma supernatants are screened by ELISA against the mixed peptide immunogen. Samples that are ELISA negative are abandoned. Strongly positive samples are screened for their ability to bind viral envelope. Using this approach a panel of monoclonal antibodies is generated against the gp41 six-helix bundle.

### EXAMPLE 2

### Assay to Detect Viral Inactivating Agents

H9 cells expressing the HIV-1 envelope proteins are resuspended in Stain/Wash Buffer (1% bovine serum albumin, 0.1% sodium azide in phosphate-buffered saline) and aliquoted at 2.5×10⁵ cells per well into a 96-well V-bottom plate containing test compounds. Negative control wells contain no test compound. Positive control wells contain recombinant soluble CD4 at a final concentration of 0.5 μg/ml. The plate is incubated for 1 hour at 37° C. to permit triggering of HIV envelope glycoprotein conformational changes. Antibody specific for the HIV gp41 six-helix bundle is then added (1 μl polyclonal serum or 1 μg monoclonal antibody per well) and the plate is incubated for an additional 1 hour at 37° C. to permit antibody binding. The cells are then washed once with Stain/Wash Buffer to remove compound and excess antibody and resuspended in DELFIA assay buffer without detergent (Perkin Elmer) containing 0.1 μg of europium-labeled anti-rabbit secondary antibody (Perkin Elmer). The cells are incubated for 45 min at 4° C. to permit secondary antibody binding. The cells are then washed twice to remove excess secondary antibody and transferred to a fresh plate. The cells are pelleted and resuspended in DELFIA enhancement solution. Time resolved fluorescence is detected using a Wallac VICTOR² multi-label plate reader (Perkin Elmer). Compounds that inactivate HIV envelope glycoprotein by triggering conformational changes that expose the six-helix bundle are identified as those that result in a significant increase in fluorescence signal due to primary antibody gaining access to the six-helix bundle epitope.

What is claimed is:

1. A method for identifying compounds that decrease the ability of a virus to infect previously uninfected cells, comprising:
provide a cell, virion, pseudovirion, membrane vesicle, lipid bilayer, or liposome expressing or bearing viral envelope protein or glycoprotein or fragment thereof,
contact said cell, virion, pseudovirion, membrane vesicle, lipid bilayer, or liposome with a candidate compound; and
measure the ability of said candidate compound to induce conformational changes in viral envelope glycoprotein or fragments thereof by determining binding of an antibody, antibody fragment or peptide to said viral envelope glycoprotein or fragments thereof.

2. The method according to claim 1, wherein said virus is a retrovirus.

3. The method of claim 1, wherein said virus is HIV.

4. The method of claim 1, wherein the ability of said candidate compound to induce conformational changes in viral envelope glycoprotein or fragments thereof, is measured by determining binding of an antibody or antibody fragment to the induced conformations.

5. The method of claim 4, wherein said antibody is either a monoclonal or polyclonal antibody.

6. The method of claim 1, wherein the antibody or antibody fragment used to measure the ability of said candidate compound to induce conformational changes in viral envelope glycoprotein or fragments thereof, comprise single chain, light chain, heavy chain, CDR, F(ab′)₂, Fab, Fab′, Fv, sFv or dsFv or any combination thereof.

7. The method of claim 1, wherein the ability of said candidate compound to induce conformational changes in viral envelope glycoprotein or fragments thereof, is measured by determining binding of a peptide to the induced conformations.

8. The method of claim 1, wherein the antibody, antibody fragment or peptide is labeled with a labeling agent.

9. The method of claim 8, wherein said labeling agent is an enzyme, fluorescent substance, chemiluminescent substance, horseradish peroxidase, alkaline phosphatase, biotin, avidin, electron dense substance, or radioisotope, or combinations thereof.

10. The method of claim 1, wherein said contact of said cell, virion, pseudovirion, membrane vesicle, or lipid bilayer with a candidate compound optionally occurs in the presence of one or more cellular receptors or fragments thereof for said virus.

11. The method of claim 10, wherein said cellular receptors are selected from the group consisting of CD4, fragments of CD4, chemokine receptors, and combinations thereof.

12. The method of claim 10, wherein said cellular receptor is soluble CD4 or membrane bound CD4.

13. The method of claim 10, wherein said cellular receptor is CCR5 or CXCR4.

14. The method according to claim 1, wherein said ability of the candidate compound to induce conformational changes is measured by incubating said cell, virion, pseudovirion, membrane vesicle, lipid bilayer, or liposome expressing or bearing viral envelope protein or glycoprotein or fragment thereof and said candidate compound with specific antibodies to determine whether the amount of antibody binding to an induced conformation necessary for viral entry is increased or decreased due to the presence of the candidate compound.

15. The method according to claim 14, wherein measuring said ability of the candidate compound to induce a change in conformation is performed by:
add one or more optionally detectably-labeled antibodies that preferentially bind an epitope that is present in a conformation or structure formed during virus entry; and
measure the amount of antibody binding.

16. The method according to claim 15, further comprising: compare the measured amount of antibody binding to a standard value.

17. The method according to claim 16, wherein said measuring the amount of antibody binding is performed by immunoprecipitation analysis, flow cytometry, fluorescence microscopy, fluorimetry, enzyme immunoassay, radiolabeling, or chemiluminescence techniques.

18. The method of claim 17, wherein the said measuring the amount of antibody binding is performed by incubating said antibody with a europium-labeled anti-rabbit secondary antibody, and detecting said secondary antibody with time resolved fluorescence.

19. The method of claim 1, wherein said ability of candidate compound to induce conformational changes in HIV envelope glycoprotein or fragments thereof is measured by detecting the presence of gp120 using antibodies to gp120 to determine whether the candidate compound has caused loss of gp120 from the surface of a cell, virion, pseudovirion, membrane vesicle, lipid bilayer, or liposome expressing or bearing viral envelope protein or glycoprotein or fragment thereof.

20. The method according to claim 1, wherein the viral envelope protein or glycoprotein is from HIV-1, HIV-2, HTLV-I, HTLV-II, respiratory syncytial virus (RSV), parainfluenza virus type 3 (HPIV-3), human influenza viruses, measles virus, hepatitis B virus (HBV) or hepatitis C virus (HCV).

21. A method for identifying compounds that decrease the ability of HIV-1 to infect previously uninfected cells, comprising:
provide HIV-1 envelope glycoproteins gp120/gp41 or fragments thereof in association with a cell, virion, pseudovirion, membrane vesicle, lipid bilayer, or liposome;
contact said HIV-1 envelope glycoproteins gp120/gp41 or fragments thereof with a candidate compound; and
measure the ability of said candidate compound to induce changes that result in the formation of entry structures in gp41.

22. The method according to claim 21, wherein said measuring step is performed by:
adding one or more optionally detectably-labeled antibodies that bind an epitope that is a structure or conformation formed during virus entry;
and measuring the amount of antibody binding.

23. The method according to claim 22, wherein said measuring the amount of antibody binding is performed by immunoprecipitation analysis, flow cytometry, fluorescence microscopy, or fluorimetry, enzyme assays, radiolabeling or chemiluminescense techniques.

24. The method of claim 21, wherein the ability of said candidate compound to induce said conformational changes in gp41 is detected by using polyclonal and/or monoclonal sera raised against peptides, a mixture of peptides, or proteins mimicking gp41 conformational structures.

25. The method of claim 24, wherein the conformational change results in a gp41 six-helix bundle structure.

26. The method of claim 24, wherein the ability of said candidate compound to induce said conformational changes in gp41 is detected by using polyclonal sera generated by immunizing animals with a 1:1 mixture of the P15 and P16 peptides.

27. The method of claim 24, wherein the ability of said candidate compound to induce said conformational changes in gp41 is detected by using monoclonal antibodies including, T26, 17b, 48d, 8F101 or A32, or mixtures thereof.
